(19) United States
(12) Patent Application Publication
(10) Pub. No.: US 2013/0012709 A1
(43) Pub. Date: Jan. 10, 2013

(54) NOVEL INHIBITORS OF STEAROYL-CoA-DESATURASE-1 AND THEIR USES

(75) Inventors: Lluis Fajas, Lausanne (CH); Zohra Benfodda, Montpellier (FR); Vanessa Fritz, Montpellier (FR)

(73) Assignee: CENTRE NATIONAL DE LA RECHERCHE SCIENTIFIQUE, Paris (FR)

(21) Appl. No.: 13/394,450

(22) PCT Filed: Sep. 10, 2010
(86) PCT No.: PCT/IB10/54092

§ 371 Date: Sep. 19, 2012

(30) Foreign Application Priority Data
Sep. 10, 2009 (EP) 09290698.1

(51) Int. Cl. C07D 295/26; C07D 403/12; C07D 409/06; C07D 401/06; C07D 417/06; C07D 409/12; C07D 405/12

(52) U.S. Cl. 544/365; 544/383; 544/379; 544/370; 544/369

(57) ABSTRACT

An inhibitor of the activity of stearoyl-CoA-desaturase-1 (SCD-1) enzyme for use in the treatment of prostate cancer as well as novel inhibitors of formula (IIa).

### FIELD OF THE INVENTION

The present invention relates to the therapy of cancers and more specifically of prostate cancer (PC). The present invention more precisely deals with the use of some piperazine derivatives for their activity against prostate cancer cells.

### BACKGROUND OF THE INVENTION

Prostate cancer (PC) is the most commonly diagnosed cancer and the second leading cause of cancer death in western men after middle-age, and remained a major research and public health priority.

With about 40,000 new cases and 10,000 deaths per year in France, PC has remained a major research and public health priority. Consistent with the trend towards an aging global population, the incidence of the disease is increasing worldwide to an average rate of 3% a year, and affects 10% of subjects beyond 70 years of age.

Bone is the main site of metastasis in PC, and up to 90% of affected patients in later stage develop skeletal metastases that are associated with significant morbidity and mortality. With almost two millions men battling prostate cancer today and the three million more predicted in the next decade, interventions to prevent, treat and avoid prostate cancer and its complications are of great economic and social impact.

Among men with early, organ-confined, PC, primary treatment with radical prostatectomy or radiotherapy in patients demonstrates overall 10-year survival rates of over 75%. However, it is estimated that approximately 40% of patients will relapse after definitive local therapy. In these patients, the use of additional therapies is required.

Because the growth of PC is initially androgen-dependent, numbers of pharmaceutical groups have been positioned in androgen deprivation therapeutic approach. Thus, over 90% of patients who failed radiotherapy undergo androgen deprivation therapy (ADT). Thus, a number of pharmaceutical groups are positioned in therapeutic approaches attempt to interrupt the production and/or the action of testosterone, with the development of Luteinizing hormone-releasing hormone (LHRH) agonists, such as Leuprolide (Bayer AG, Sanofi-Aventis, TAP Pharmaceuticals), Goserelin (AstraZeneca), Triptorelin (Ipsen, Ferring Pharmaceuticals, Pfizer), or to block the action of testosterone by using anti-androgens, such as Bicalutamide (AstraZeneca) and Flutamide (Schering-Plough).

However, despite an initial response to ADT, ultimately all patients with advanced PC experience disease progression and invariably become refractory to hormonal manipulation within 18-24 months. Historically, once men reach this stage, therapeutic options are limited and prognosis is poor, with a median survival time of 12-16 months. Moreover, up to 90% of patients with advanced PC are affected by skeletal metastasis, an incurable progression of the disease that accounts for the vast majority of disease-related mortality and its associated morbidity. Although systemic therapy has proven effective for many cancers, currently used chemotherapeutic agents are not curative in the treatment of androgen-independent prostate cancer (AIPC). Indeed, no single chemotherapeutic agent such as Mitoxantrone (Merk), Docetaxel (Sanofi-Aventis), Ixabepilone (Bristol-Myers Squibb), or combination of agents with corticosteroids, has been shown to prolong life.

Nevertheless, despite an initial response to treatment, most patients with advanced disease eventually develop resistance and progress to hormone refractory PC for which there is currently no curative therapy. Although many efforts have been directed towards more efficient ways to ablate the androgen action, little attention has been paid to the metabolism of prostate cancer cells.

In view of this, there is currently no curative treatment of prostate cancer and new therapeutic options are expected.

### SUMMARY OF THE INVENTION

The present invention raises from the unexpected observation of the inventors that inhibitors of SCD-1 activity are potent new therapeutic agent to inhibit PC disease.

High ratio of monounsaturated to saturated fatty acids affect phospholipids composition and has been correlated with neoplastic transformation, as demonstrated by FA analysis of lipids extracts from transformed cells, cancerous cells and tumor tissue (Apostolov et al., Blut, 1985, 50:349; Wood et al., Eur. J. Surg. Oncol., 1985, 11:347). Moreover, studies have evidenced that stearic acid treatment leads to inhibition of tumor cell growth both in vitro and in vivo (Habib et al., Br. J. Cancer, 1987, 56:455).

Unexpectedly, the inventors have observed that inhibition of SCD-1 activity altered lipid synthesis and proliferation in prostate cancer cell lines.

In addition, as shown in the following examples, by using siRNA, they observe that specific silencing hSCD-1 gene expression significantly reduces proliferation in both androgen-sensitive LNCaP and androgen-insensitive C4-2 prostate cancer cell lines.

Consequently, in one aspect, the present invention relates to inhibitors of the expression and/or activity of human stearyl-CoA-desaturase (hSCD) enzymes, especially of stearyl-CoA-desaturase-1 (SCD-1) for use in the treatment of prostate cancer.

Accordingly to one embodiment, the prostate cancer is a prostate cancer with a Gleason score equal or superior to 7.

According to one embodiment, the instant invention relates to the use of inhibitors of the expression of SCD-1.

According to another embodiment, the instant invention relates to the use of inhibitors of the expression of SCD-1 selected from the group consisting of antisense RNA or DNA molecules, small interfering RNAs (siRNAs), short hairpin RNAs (shRNAs) and ribozymes.

According to another embodiment, the instant invention relates to the use of inhibitors of the activity of SCD1.

According to another embodiment, the instant invention relates to the use of inhibitors of the activity of SCD1 of formula (I):

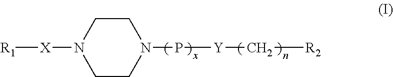

wherein X, R₁, P, x, Y, n and R₂ are as defined hereafter.

According to another aspect, the instant invention relates to inhibitors of the expression and/or activity of human stearyl-CoA-desaturase (hSCD) enzymes, especially stearyl-CoA-desaturase-1 (SCD-1), and in particular of compounds of the formula (I), for use as selective agent for blocking prostate cancer (PC) cells proliferation

The present invention also relates to a use of an inhibitor of the expression and/or activity of human stearyl-CoA-desaturase (hSCD) enzymes, especially stearyl-CoA-desaturase-1 (SCD-1), and in particular of compounds of the formula (I), as selective agent for blocking prostate cancer (PC) cells proliferation for the preparation of a pharmaceutical composition for treating prostate cancer.

In another aspect, the invention relates to a method for treating a prostate cancer, comprising the administration to a patient in need thereof of an effective amount of at least one inhibitor of the expression and/or activity of human SCD enzymes, especially SCD-1, and in particular of compounds of the formula (I).

According to another aspect, the invention relates to compounds of formula (II) as defined hereafter.

In a another aspect, the invention relates to a pharmaceutical composition comprising an effective amount of at least a compound of formula (II) as defined hereafter, optionally in combination with at least one cancer agent different from said compound of formula (II), and in particular a secondary chemotherapeutic agent.

According to another aspect, the present invention relates to a pharmaceutical composition containing as active agent at least one compound according to the invention, and in particular of formula (IIa) as defined hereafter.

A pharmaceutical composition of the invention comprises an active compound in a therapeutically effective amount.

Such compound of formula (II) and/or pharmaceutical composition containing at least one of them are particularly useful for preventing and/or treating SCD-mediated disease. These diseases are detailed hereafter.

Due to its role in lipid metabolism, SCD-1 is a therapeutic target for the treatment of diseases related to metabolic syndrome, including but not limited to cancer, acnea, obesity and obesity-related diseases, that is hypertension, insulin resistance, diabetes, atherosclerosis and heart failure.

An SCD-mediated disease or condition includes metabolic syndrome (including but not limited to dyslipidemia, obesity and insulin resistance, hypertension, microalbuminemia, hyperuricaemia, and hypercoagulability), Syndrome X, diabetes, insulin resistance, decreased glucose tolerance, non-insulin-dependent diabetes mellitus, Type II diabetes, Type I diabetes, diabetic complications, body weight disorders, weight loss, body mass index and leptin related diseases.

An SCD-mediated disease or condition also includes fatty liver, hepatic steatosis, hepatitis, non-alcoholic hepatitis, non-alcoholic steatohepatitis (NASH), alcoholic hepatitis, acute fatty liver, fatty liver of pregnancy, drug-induced hepatitis, erythrohepatic protoporphyria, iron overload disorders, hereditary hemochromatosis, hepatic fibrosis, hepatic cirrhosis, hepatoma and conditions related thereto.

An SCD-mediated disease or condition also includes but is not limited to a disease or condition which is, or is related to primary hypertriglyceridemia, or hypertriglyceridemia secondary to another disorder or disease, such as hyperlipoproteinemias, familial histiocytic reticulosis, lipoprotein lipase deficiency, apolipoprotein deficiency (such as ApoC II deficiency or ApoE deficiency), and the like, or hypertriglyceridemia of unknown or unspecified etiology.

An SCD-mediated disease or condition also includes a disorder of polyunsaturated fatty acid (PUFA) disorder, or a skin disorder, including but not limited to eczema, acne, psoriasis, keloid scar formation or prevention, diseases related to production or secretions from mucous membranes, such as monounsaturated fatty acids, wax esters, and the like. An SCD-mediated disease or condition also includes inflammation, sinusitis, asthma, pancreatitis, osteoarthritis, rheumatoid arthritis, cystic fibrosis, and pre-menstrual syndrome.

An SCD-mediated disease or condition also includes but is not limited to a disease or condition which is, or is related to cancer, more particularly lung cancer and prostate cancer, breast cancer, hepatomas and the like, neoplasia, malignancy, metastases, tumours (benign or malignant), carcinogenesis.

An SCD-mediated disease or condition also includes a condition where increasing lean body mass or lean muscle mass is desired, such as is desirable in enhancing performance through muscle building. Myopathies and lipid myopathies such as carnitine palmitoyltransferase deficiency (CPT I or CPT II) are also included herein. Such treatments are useful in humans and in animal husbandry, including for administration to bovine, porcine or avian domestic animals or any other animal to reduce triglyceride production and/or provide leaner meat products and/or healthier animals.

An SCD-mediated disease or condition also includes a disease or condition which is, or is related to, neurological diseases, psychiatric disorders, multiple sclerosis, eye diseases, and immune disorders.

### DEFINITIONS

“Therapeutically effective amount” refers to an amount of a compound of the invention which, when administered to a mammal, preferably a human, is sufficient and necessary to effect a treatment, as defined below, in the mammal, preferably a human. The amount of a compound of the invention which constitutes a “therapeutically effective amount” will vary depending on the compound, the condition and its severity, and the age of the mammal to be treated, but can be determined routinely by one of ordinary skill in the art having regard to his own knowledge and to this disclosure.

“Treating” or “treatment” as used herein covers the treatment of the disease or condition of interest in a mammal, preferably a human, having the disease or disorder of interest, and includes:
(i) preventing the disease or condition from occurring in a mammal, in particular, when such mammal is predisposed to the condition but has not yet been diagnosed as having it;
(ii) inhibiting the disease or condition, i.e., arresting its development; or
(iii) relieving the disease or condition, i.e., causing regression of the disease or condition.

The “prophylactic and/or therapeutic agent” as hereunder mentioned may be a compound according to the invention itself having a prophylactic and/or therapeutic action on indicated diseases or a pharmaceutical agent containing such a substance.

As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

According to the present invention, the terms below have the following meanings

The term “halogen atom” corresponds to a fluorine, chlorine, bromine or iodine atom.

The term “alkyl” as used herein refers to a saturated, linear or branched aliphatic group. The following examples may be cited: methyl, ethyl, 1-propyl, 2-propyl, 1-butyl, 2-methyl-1-propyl (also named i-Bu), 2-butyl (also named s-Bu), 2-methyl-2-propyl (also named t-Bu), 1-pentyl (also named n-pentyl), 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, n-pentyl, n-hexyl. Preferred alkyl according to the invention are methyl, ethyl, 1-propyl, 2-propyl, 1-butyl, 2-methyl-1-propyl (also named i-Bu), 2-butyl (also named s-Bu), 2-methyl-2-propyl (also named t-Bu), 1-pentyl (also named n-pentyl), 2-pentyl, 3-pentyl, 2-methyl-2-butyl, 3-methyl-2-butyl, 3-methyl-1-butyl, 2-methyl-1-butyl.

As used herein and unless otherwise stated, the term “cycloalkyl” means a saturated cyclic alkyl group as defined above. The following examples may be cited: cyclopropyl, methylcyclopropyl, cyclobutyl, methylcyclobutyl, methylcyclopentyl, cyclopentyl, cyclohexyl. Preferred cycloalkyl according to the invention are cyclopentyl or cyclohexyl.

The term “alkenyl” corresponds to a linear or branched, unsaturated aliphatic group, comprising at least one unsaturation site (usually 1 to 3 and preferably 1), i.e. a carbon-carbon sp2 double bound. The following examples may be cited: ethylene, allyl. The double bond may be in the cis or trans configuration.

The term “alkoxy” corresponds to a —O-alkyl group, wherein the alkyl group is as defined above. The following examples may be cited: methoxy, ethoxy, propoxy.

The term “aryl” as used herein means an aromatic mono- or poly-cyclic group in C₅ to C₁₄. An example of monocyclic group may be phenyl. Examples of polycyclic rings may be naphthalene, anthracene, and biphenyl.

The term “heterocyclyl” or “heterocycloalkyl” as used herein refers to a cycloalkyl in C₅ to C₁₄ as described above further comprising at least one heteroatom chosen from nitrogen (N), oxygen (O), or sulphur (S) atom. The following examples may be cited: piperidinyl, piperazinyl, morpholinyl, 1,4-dioxanyl, 1,4-dithianyl, homomorpholinyl, 1,3,5-trithianyl, pyrrolidinyl, 2-pyrrolidinyl, tetrahydro furanyl, tetrahydropyranyl.

The term “heteroaryl” as used herein corresponds to an aromatic, mono- or poly-cyclic group comprising between 5 and 14 carbon atoms and comprising at least one heteroatom such as nitrogen, oxygen or sulphur atom. Examples of such mono- and poly-cyclic heteroaryl group may be: pyridyl, thiazolyl, thiophenyl, furanyl, pyranyl, pyrrolyl, pyrazolyl, imidazolyl, tetrazolyl, benzofuranyl, thianaphthalenyl, indolyl, quinolinyl, isoquinolinyl, benzimidazolyl, pyrrolinyl, isothiazolyl, isoxazolyl, pyrazinyl, pyridazinyl, indolizinyl, isoindolyl, phthalazinyl, naphthyridinyl, quinoxalinyl, quinazolinyl, imidazo linyl, pyrazo lidinyl, pyrazo linyl, indolinyl, iso indo linyl, oxazolidinyl, benzotriazolyl, benzisoxazolyl, oxindolyl, benzoxazolinyl, benzothienyl, benzothiazolyl, pyridinyl, dihydropyridyl, pyrimidinyl, pyrazinyl, oxazolyl, thiofuranyl (thiophenyl or thienyl).

Rings as defined above, comprising at least one heteroatom, may be bound through a carbon atom or a heteroatom.

Regardless of bond indications, if a substituent is polyvalent (based on its position in the structure referred to, then any and all possible orientations of the substituent are intended.

### DETAILED DESCRIPTION OF THE INVENTION

The compounds considered according to the invention as active against the PC are capable of inhibiting the expression and/or activity of the human SCD-1 enzyme.

According to one embodiment, an inhibitor preferably considered in the invention may be an inhibitor of the expression of SCD-1.

In another preferred embodiment, an inhibitor of the expression of the SCD-1 more particularly considered in the invention may be selected from the group consisting of antisense RNA or DNA molecules, small interfering RNAs (siRNAs), short hairpin RNAs (shRNAs) and ribozymes.

RNA interference is a post-transcriptional process observed in various organisms whereby double-stranded RNA molecules mediate gene silencing in a sequence-specific manner. RNA interference may be carried out using short interfering RNAs (siRNAs), which are generally about 19-22 nucleotides long.

As example of siRNA that may be used in the instant invention one may mention SEQ ID NO: 1 GCACAUCAACUUCACCACA.

A small hairpin RNA or short hairpin RNA (shRNA) is a sequence of RNA that makes a tight hairpin turn that can be used to silence gene expression via RNA interference.

Antisense polynucleotides (such as antisense RNAs or DNAs) may be used to suppress expression of a gene, either before or after transcription. Antisense polynucleotide is a polynucleotide which contains a stretch of nucleotides complementary to another polynucleotide (RNA or DNA) that has some cellular function. The length of the complementary stretch is usually a few hundred nucleotides, but shorter stretches can also be used.

As examples of method useful in the invention for obtaining antisense RNAs or antisense DNAs, siRNA or shRNAs, one may mention the methods described in WO 2006/060454, WO 2001/025488, WO 2004/108897, or US 2006/223777.

Ribozymes are synthetic RNA molecules having highly specific endoribonuclease activity. A ribozyme comprises a hybridizing region which is complementary in nucleotide sequence to at least part of a target RNA, and a catalytic region which is adapted to cleave the target RNA. Methods that may be used for obtaining ribozymes in accordance with the invention are, for example, described in U.S. Pat. No. 5,494,814 or EP 0 321 201.

On the basis of the nucleic acid sequence encoding for the human SCD-1 (NCBI Reference Sequence NM_{—}005063.4) and on its knowledge in the field, a man skilled in the art may readily obtain antisense RNA or DNA molecules, small interfering RNAs (siRNAs), short hairpin RNAs (shRNAs) and ribozymes suitable for the invention.

According to another embodiment, an inhibitor preferably considered in the invention may be an inhibitor of the activity of SCD-1.

The determination of inhibiting the activity of human SCD1 enzyme may be readily accomplished using the SCD-1 enzyme and microsomal assay procedure described in Brownlie et al., (WO 01/62954).

More particularly these SCD-1 inhibitors may be selected from the group consisting of:
pyridine derivatives as disclosed in WO 2005/011654 and WO 2005/011656 and in particular the following compounds,

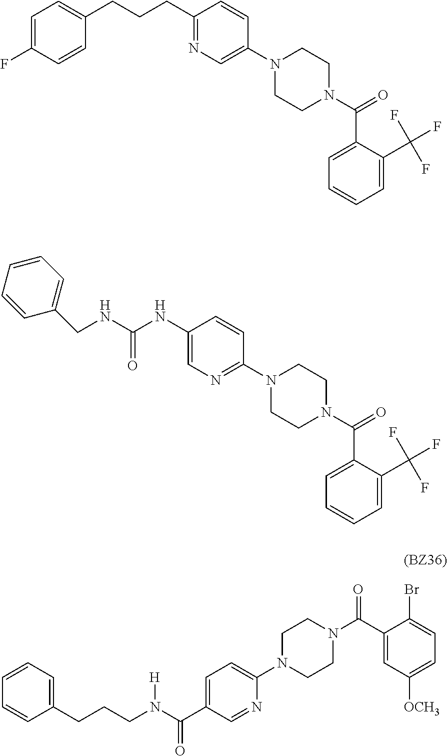 [SMILES: O=C(c1ccccc1C(F)(F)F)N1CCN(c2ccc(CCCc3ccc(F)cc3)nc2)CC1 ; O=C(NCc1ccccc1)Nc1ccc(N2CCN(C(=O)c3ccccc3C(F)(F)F)CC2)nc1 ; COc1ccc(Br)c(C(=O)N2CCN(c3ccc(C(=O)NCCCc4ccccc4)cn3)CC2)c1]

pyridazines derivatives as disclosed in WO 2005/011655 and WO 2006/086447 and in particular the following compound,

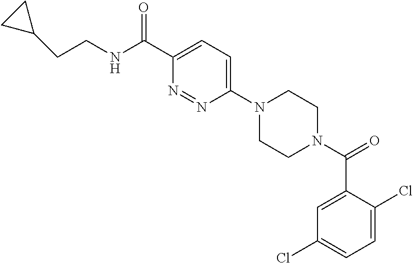 [SMILES: O=C(NCCC1CC1)c1ccc(N2CCN(C(=O)c3cc(Cl)ccc3Cl)CC2)nn1]

piperidine derivatives as the following compound,

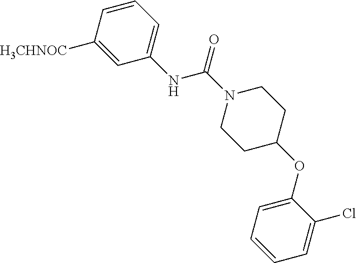

nicotinamides derivatives as disclosed in WO 2006/014168,
heterocyclic bicyclic derivatives as disclosed in WO 2006/034312 and in particular the following compound,

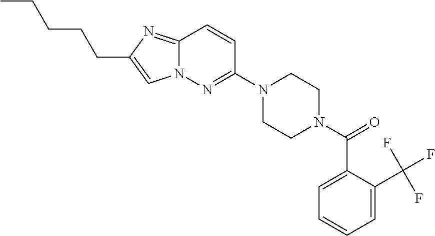 [SMILES: CCCCCc1cn2nc(N3CCN(C(=O)c4ccccc4C(F)(F)F)CC3)ccc2n1]

heterocyclic derivatives as disclosed in WO 2006/034315, WO 2006/034338, WO 2006/034446, WO 2006/101521 and WO 2006/034441, and in particular the following compounds,

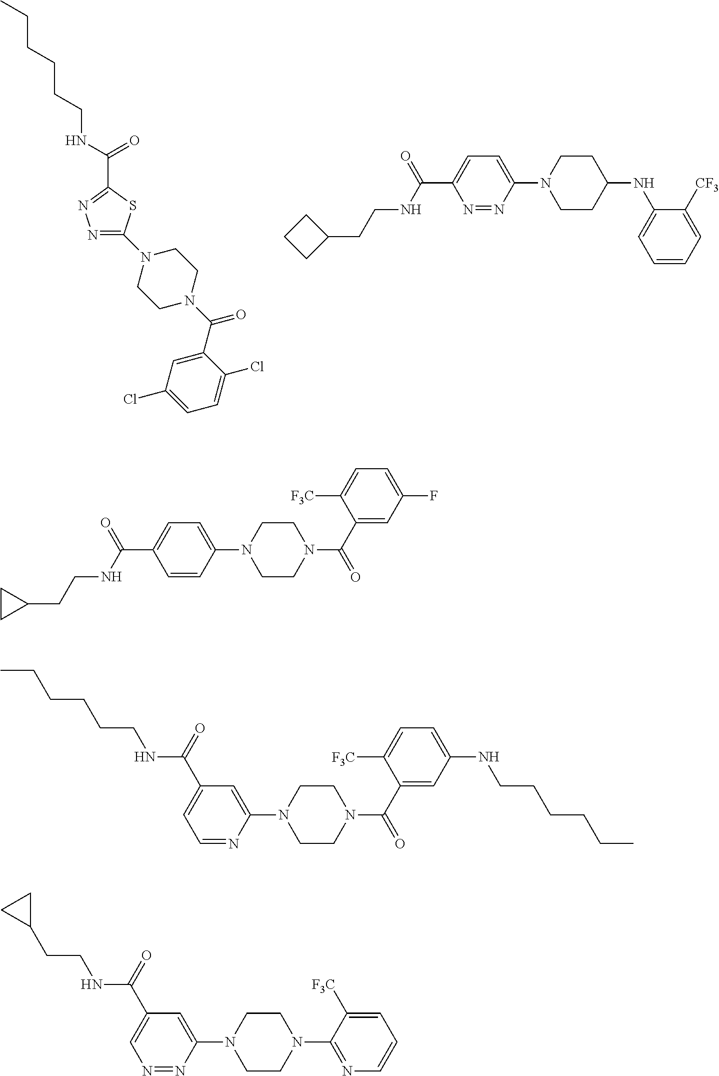 [SMILES: CCCCCCNC(=O)c1nnc(N2CCN(C(=O)c3cc(Cl)ccc3Cl)CC2)s1 ; O=C(NCCC1CCC1)c1ccc(N2CCC(Nc3ccccc3C(F)(F)F)CC2)nn1 ; O=C(NCCC1CC1)c1ccc(N2CCN(C(=O)c3cc(F)ccc3C(F)(F)F)CC2)cc1 ; CCCCCCNC(=O)c1ccnc(N2CCN(C(=O)c3cc(NCCCCCC)ccc3C(F)(F)F)CC2)c1 ; O=C(NCCC1CC1)c1cnnc(N2CCN(c3ncccc3C(F)(F)F)CC2)c1]

piperazines derivatives as disclosed in WO 2006/125180, and in particular the following compounds,

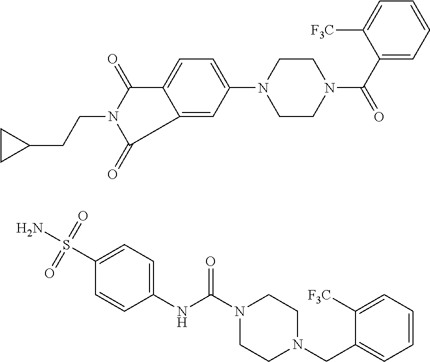 [SMILES: O=C(c1ccccc1C(F)(F)F)N1CCN(c2ccc3c(c2)C(=O)N(CCC2CC2)C3=O)CC1 ; NS(=O)(=O)c1ccc(NC(=O)N2CCN(Cc3ccccc3C(F)(F)F)CC2)cc1]

heteroaryl derivatives as disclosed in WO 2007/044085, and in particular the following compound,

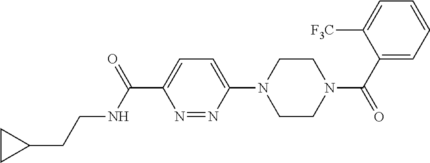 [SMILES: O=C(NCCC1CC1)c1ccc(N2CCN(C(=O)c3ccccc3C(F)(F)F)CC2)nn1]

thiazolidines derivatives as disclosed in WO 2007/046868, and in particular the following compound,

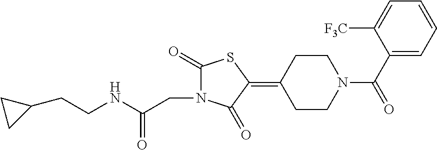 [SMILES: O=C(CN1C(=O)SC(=C2CCN(C(=O)c3ccccc3C(F)(F)F)CC2)C1=O)NCCC1CC1]

piperidines derivatives as disclosed in WO 2007/050124, and in particular the following compound,

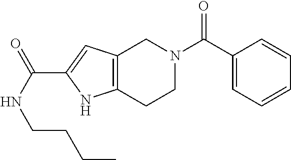 [SMILES: CCCCNC(=O)c1cc2c([nH]1)CCN(C(=O)c1ccccc1)C2]

aza cyclohexane derivatives as disclosed in WO 2007/056846, and in particular the following compound,

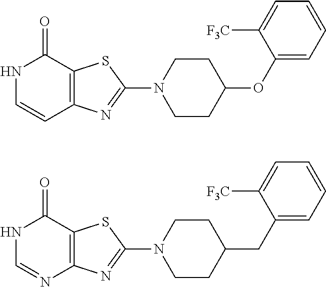 [SMILES: O=c1[nH]ccc2nc(N3CCC(Oc4ccccc4C(F)(F)F)CC3)sc12 ; O=c1[nH]cnc2nc(N3CCC(Cc4ccccc4C(F)(F)F)CC3)sc12]

heteroaromatic derivatives as disclosed in WO 2007/071023 and in the publication from J. Med. Chem., 2007, 50, 13, 3086-3100, and in particular the following compounds,

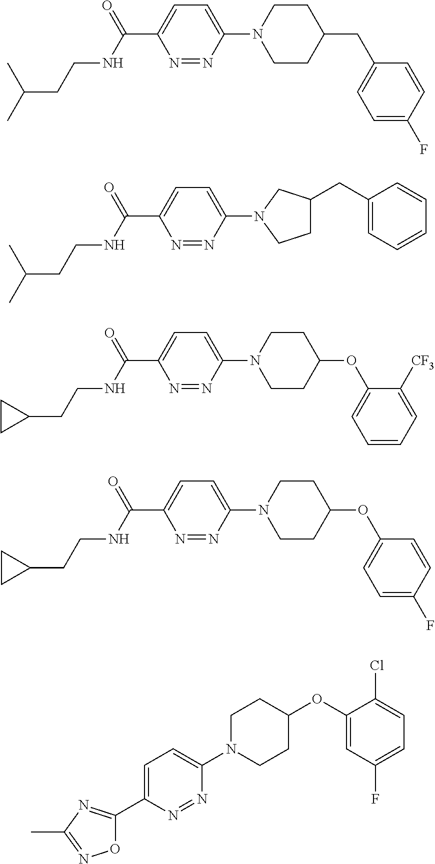

bicyclic heteroaromatic derivatives as disclosed in WO 2009/012573, and in particular the following compounds,

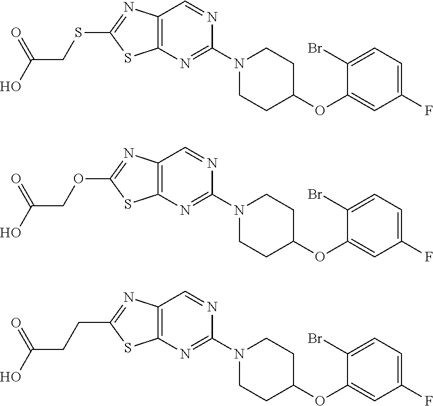 [SMILES: O=C(O)CSc1nc2cnc(N3CCC(Oc4cc(F)ccc4Br)CC3)nc2s1 ; O=C(O)COc1nc2cnc(N3CCC(Oc4cc(F)ccc4Br)CC3)nc2s1 ; O=C(O)CCc1nc2cnc(N3CCC(Oc4cc(F)ccc4Br)CC3)nc2s1]

According to another preferred embodiment, an inhibitor of activity of the SCD-1 may be a compound represented by the following formula (I):

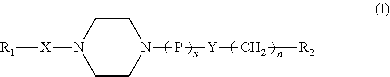

wherein
R₁ represents an alkyl, a cycloalkyl, an aryl or a heteroaryl group in C₅ to C₁₄, in particular in C₆, said aryl or heteroaryl being optionally substituted with one or more groups Rₐ;
Rₐ represents an halogen atom, an hydroxyl group, —NO₂, —CN, —NH₂, —N(C₁₋₆alkyl)₂, a C₁₋₆alkyl, a C₁₋₆alkoxy, a —C(O)—C₁₋₆alkyl, C₂₋₆alkenyl, C₃₋₆cycloalkyl, aryl, C₃₋₆ heterocyclyl or heteroaryl, said alkyl, alkoxy, alkenyl, cycloalkyl, aryl, heterocyclyl or heteroaryl being optionally substituted with one or more halogen atom, C₁₋₆ alkyl, C₁₋₆ alkoxy, —C(O)—C₁₋₆ alkyl, —NO₂, —CF₃, —OCF₃, —CN, —NH₂, and/or —N(C₁₋₆alkyl)₂;
P is a heteroaromatic cycle in C₅ to C₁₄, in particular in C₆;
x represents 0 or 1;
Y represents —SO₂— or *—CO—NH—, *—CS—NH—, with * figuring the link to —(P)ₓ—;
n represents 0, 1, 2 or 3;
R₂ represents an alkyl, a cycloalkyl, an aryl or a heteroaryl group in C₅ to C₁₄, in particular in C₆, said aryl or heteroaryl being optionally substituted with one or more groups R_{b};
R_{b} represents an halogen atom, an hydroxyl group, NO₂, —CN, —CF₃, —OCF₃, a C₁₋₆ alkyl, C₁₋₆ alkoxy, —C(O)—C₁₋₆ alkyl, —NH₂, —N(C₁₋₆alkyl)₂, C₃₋₆ cycloalkyl, C₃₋₆ heterocyclyl, aryl, heteroaryl, said alkyl, alkoxy, cycloalkyl, heterocyclyl, aryl, heteroaryl being optionally substituted with one or more hydroxyl group, —CF₃, —OCF₃, —NH₂, —NO₂, and/or —CN,
   or one of its salts or enantiomer forms.

The compounds of formula (I) can comprise one or more asymmetrical carbon atoms. They can thus exist in the form of enantiomers or of diastereoisomers. These enantiomers, diastereoisomers, or their mixtures, including the racemic mixtures form part of the invention.

The compounds of the invention may exist in the form of free bases or of addition salts with pharmaceutically acceptable acids.

Suitable pharmaceutically acceptable salts of compounds of formula (I) include base addition salts and where appropriate acid addition salts. Suitable physiologically acceptable addition salts of compounds (I) include alkali metal or alkaline metal salts such as sodium, potassium, calcium, and magnesium salts, and ammonium salts, formed with amino acids (e.g. lysine and arginie) and organic bases (e.g. procaine, phenylbenzylamine, ethanolamine diethanolamine and N-methyl glucosamine).

Suitable acid addition salts may be formed with organic acid and inorganic acids, e.g. hydrochloric acid.

The compounds of formula (I) can also exist in the form of a hydrate or of a solvate, i.e. in the form of associations or combinations with one or more water or solvent molecules. Such hydrates and solvates also form part of the invention.

According to a specific embodiment in the above-defined compounds of formula (I) of the present invention, X represents —CO—.

According to another specific embodiment in the above-defined compounds of formula (I) of the present invention, R1 is an aryl or a heteroaryl group in C₅ to C₁₄, in particular in C₆, said aryl or heteroaryl being optionally substituted with one or more groups Rₐ.

According to another specific embodiment in the above-defined compounds of formula (I) of the present invention, Y represents —SO₂— or *—CO—NH—, with * figuring the link to —(P)ₓ—.

According to a preferred variant, an SCD inhibitor may be the 1N-[(2-bromo-5-methoxybenzoyl)]-4N-[phenylpropan)pyridine-3-carboxamide]piperazine (BZ36).

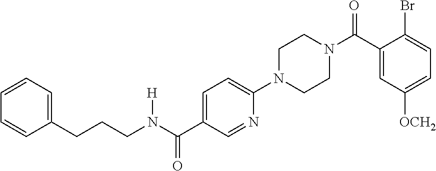

or one of its salts or enantiomer forms.

As shown hereafter, treatment with the 1N-[(2-bromo-5-methoxybenzoyl)]-4N-[(phenylpropan)pyridine-3-carboxamide)]piperazine significantly inhibits the growth of PC cell lines both in vitro and in vivo in subcutaneous xenografts mouse models of PC.

According to another preferred variant, a compound considered according to the invention may be of general formula (II):

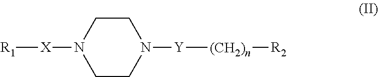

wherein X, Y, R₁, Rₐ, R₂, R_{b} and n are as defined previously.

The compounds of formula (II) form also part of the present invention as new compounds.

More particularly, the compound of formula (II) may be represented by one of the following formula:

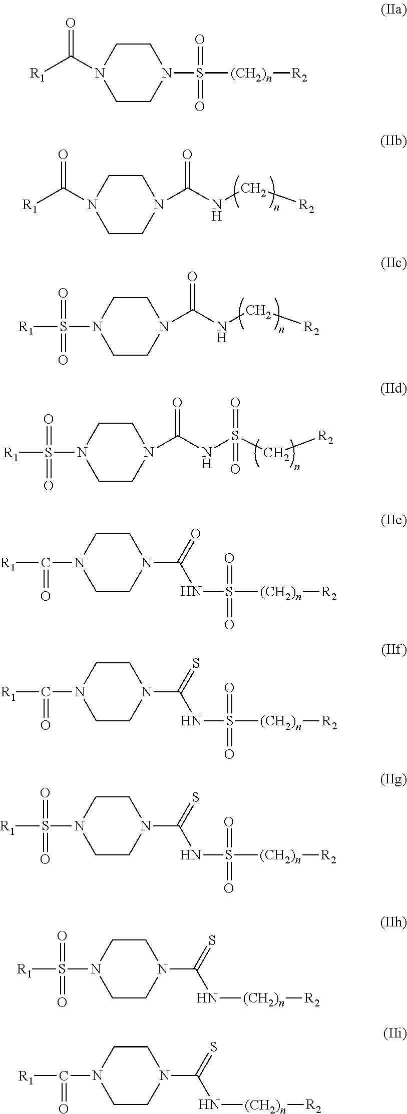

with R₁, n and R₂ being as defined previously.

Among the compound of formula (IIa) to (IIi) here-above defined, mention may be more particularly made to the compounds of formula (IIa).

A first class of compounds of formula (IIa) is defined with R₁ representing a phenyl group, optionally substituted with one or more Rₐ, said Rₐ representing an halogen atom or a C₁₋₆alkoxy group, and more particularly a bromo, methoxy a benzoyl group; and n represents 0 or 1.

According to a specific embodiment, R₁ is a 2-bromo-5-methoxybenzoyl group.

A second class of compounds of formula (IIa) is defined with R₁—(C═O) representing a heteroarylcarbonyl group selected form the group consisting of nicotinoyl, thiophene carbonyle, and thiazolecarbonyle.

According to a specific embodiment in the above-defined compounds of formula (II) and (IIa), R₂ may preferably be a difluorophenyl group.

According to another specific embodiment in the above-defined compounds of formula (IIa):

R₁ represents an alkyl, an aryl or a heteroaryl group in C₅ to C₁₄, in particular in C₆, said aryl or heteroaryl being optionally substituted with one or more groups Rₐ;

Rₐ represents an halogen atom, an hydroxyl group, —NO₂, —CN, —NH₂, —N(C₁₋₆alkyl)₂, a C₁₋₆alkyl, a C₁₋₆alkoxy, a —C(O)—C₁₋₆alkyl, C₂₋₆alkenyl, C₃₋₆cycloalkyl, aryl, C₃₋₆ heterocyclyl or heteroaryl, said alkyl, alkoxy, alkenyl, cycloalkyl, aryl, heterocyclyl or heteroaryl being optionally substituted with one or more halogen atom, C₁₋₆ alkyl, C₁₋₆ alkoxy, —C(O)—C₁₋₆ alkyl, —NO₂, —CF₃, —OCF₃, —CN, —NH₂, and/or —N(C₁₋₆alkyl)₂;

R₂ is a difluorophenyl group, and

n is as defined above in formula (I).

More particularity, the compounds of formula (II) considered according to the invention may be group consisting of:
1N-[(2-bromo-5-methoxybenzoyl)]-4N-[(phenyl)sulfonyl]piperazine,
1N-[(2-bromo-5-methoxybenzoyl)]-4N-[(4-methylphenyl)sulfonyl]piperazine,
1N-[(2-bromo-5-methoxybenzoyl)]-4N-[(4-propylphenyl)sulfonyl]piperazine,
1N-[(2-bromo-5-methoxybenzoyl)]-4N-[(4-isopropylphenyl)sulfonyl]piperazine,
1N-[(2-bromo-5-methoxybenzoyl)]-4N-[(4-nitrophenyl)sulfonyl]piperazine,
1N-[(2-bromo-5-methoxybenzoyl)]-4N-[(4-chlorophenyl)sulfonyl]piperazine,
1N-[(2-bromo-5-methoxybenzoyl)]-4N-[(4-bromophenyl)sulfonyl]piperazine,
1N-[(2-bromo-5-methoxybenzoyl)]-4-[(4-fluorophenyl)sulfonyl]piperazine,
1N-[(2-bromo-5-methoxybenzoyl)]-4N-[(4-methoxyphenyl)sulfonyl]piperazine,
1N-[(2-bromo-5-methoxybenzoyl)]-4N-[(4-acetylphenyl)sulfonyl]piperazine,
1N-[(2-bromo-5-methoxybenzoyl)]-4N-[(phenylmethane)sulfonyl]piperazine,
1N-[(2-bromo-5-methoxybenzoyl)]-4N-[(4-biphenyl)sulfonyl]piperazine,
1N-[(2-bromo-5-methoxybenzoyl)]-4N-[(2-fluorophenyl)sulfonyl]piperazine,
1N-[(2-bromo-5-methoxybenzoyl)]-4N-[(3-fluorophenyl)sulfonyl]piperazine,
1N-[(2-bromo-5-methoxybenzoyl)]-4N-[(4-trifluoromethylphenyl)sulfonyl]piperazine,
1N-[(2-bromo-5-methoxybenzoyl)]-4N-[(4-trifluoromethoxyphenyl)sulfonyl]piperazine,
1N-[(2-bromo-5-methoxybenzoyl)]-4N-[(3-trifluoromethylphenyl)sulfonyl]piperazine,
1N-[(2-bromo-5-methoxybenzoyl)]-4N-[(2-trifluoromethylphenyl)sulfonyl]piperazine,
1N-[(2-bromo-5-methoxybenzoyl)]-4N-[(2,4-difluorophenyl)sulfonyl]piperazine,
1N-[(2-bromo-5-methoxybenzoyl)]-4N-[(2,6-difluorophenyl)sulfonyl]piperazine,
1N-[(2-bromo-5-methoxybenzoyl)]-4N-[(2-thiophene)sulfonyl]piperazine,
1N-[(2-bromo-5-methoxybenzoyl)]-4N-[(3-thiophene)sulfonyl]piperazine,
1N-[1N-[(2-bromo-5-methoxybenzoyl)]-4N-[(3-furan)sulfonyl]piperazine,
1N-[(2-bromo-5-methoxybenzoyl)]-4N-[(2-furan)sulfonyl]piperazine,
1N-[(2-bromo-5-methoxybenzoyl)]-4N-[(1-methyl-1H-imidazo le)sulfonyl]piperazine,
1N-[(3-bromo-4-methoxybenzoyl)]-4-N-[(2,4-difluorophenyl)sulfonyl]piperazine,
1N-[(2-methoxy-5-bromobenzoyl)]-4N-[(2,4-difluorophenyl)sulfonyl]piperazine,
1N-[(2-methoxy-3-bromobenzoyl)]-4-N-[(2,4-difluorophenyl)sulfonyl]piperazine,
1N-[(4-methoxybenzoyl)]-4-N-[(2,4-difluorophenyl)sulfonyl]piperazine,
1N-[(2-methoxybenzoyl)]-4-N-[(2,4-difluorophenyl)sulfonyl]piperazine,
1N-[(3-methoxybenzoyl)]-4-N-[(2,4-difluorophenyl)sulfonyl]piperazine,
1N-[nicotinoyl]-4-N-[(2,4-difluorophenyl)sulfonyl]piperazine,
1N-[isonicotinoyl]-4-N-[(2,4-difluorophenyl)sulfonyl]piperazine,
1N-[picolinoyl]-4-N-[(2,4-difluorophenyl)sulfonyl]piperazine,
1N-[3-thiophenecarbonyl]-4-N-[(2,4-difluorophenyl)sulfonyl]piperazine,
1N-[4-thiazolecarbonyl]-4-N-[(2,4-difluorophenyl)sulfonyl]piperazine,
1N-[(4-bromobenzoyl)]-4-N-[(2,4-difluorophenyl)sulfonyl]piperazine, and their salts.

Compounds pertaining to the hereabove defined formula (IIa) form part of the invention as the pharmaceutical composition including them.

More specifically, the instant invention is also more particularly directed to the compound examples described in the following examples 3, 8, 9, 11, 12, 13 16, 17, 21, 23, 26, 28.

The compounds considered according to the invention may be easily obtained according to the following scheme for synthesis:

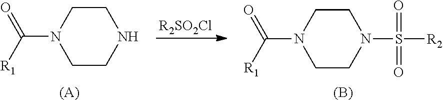

with R₁ and R₂ being as defined previously.

To a solution of (A) (1 equiv) in anhydrous solvent like for example CH₂Cl₂, substituted phenyl sulfonyl chloride (1 equiv) is added in presence of a base like, for example, N,N diisopropyl ethyl amine. The mixture was maintained under stirring until the expected reaction be completed. The compound (B) is isolated from the rectional mixture according to a conventional method.

The starting compounds are generally commercially available or can be prepared according to methods known to the person skilled in the art in particular as disclosed in the following example.

The following examples provide with details of preparation of compounds according to the invention.

### EXPERIMENTAL SECTION

Reagents were obtained from Sigma-Aldrich or Acros. Characterization of all compounds was done with ¹H, ¹⁹F, ¹³C NMR, and HRMS. ¹H, ¹⁹F, and ¹³C NMR spectra were recorded at 300.13 MHz, 282.37 MHz and 75.46 MHz respectively with a Brucker Avance 300 spectrometer, therefore chemical shifts were given in ppm relative to Me₄Si, CCl₃F respectively, as internal standards. Coupling constants were given in Hz. High Resolution Mass Spectrometry (HRMS) were recorded on a Jeol SX 102 spectrometer. High Pressure Liquid Chromatographie (HPLC) analyses were obtained on the Waters Alliance 2795 using the following conditions: thermo Hypersil C18 column (3 μm, 50 mm L×2.1 mm ID), 20° C. column temperature, 0.2 mL/min flow rate, photodiodearray detection (210-400 nm).

Melting points were recorded at atmospheric pressure unless otherwise stated on a Stuart scientific SMP3 apparatus and were remained without any correction. The products were purified by column chromatography.

Thin layer chromatography was performed with Merck Silica gel aluminium-backed plate with UV visualization. The following synthetic conditions have not been optimized.

### Preparation of 1N-(2-bromo-5-methoxybenzoyl)piperazine

In a 1000 mL flask were added 370 mL of CH₂Cl₂ and 12.63 g (147 mmol) of piperazine. The resulting solution, maintained at 0° C. using an ice bath, was added dropwise a ditert butyldicarbonate solution (16 g, 73.5 mmol in 150 mL of CH₂Cl₂). The mixture was stirred for additionnal 1 h, filtered and the filtrate concentrated to dryness. Water (220 mL), was added to the resulting oil and the mixture filtered. The filtrate was saturated with potassium carbonate and extracted with diethyl ether (3×100 mL). The solvent was dried over Na₂SO₄ and concentrated to dryness yielding to 9 g tert-butyl piperazine-1-carboxylate (66%).

A solution of so-obtained piperazine-1-carboxylic acid tert-butyl ester (8.7 g, 47 mmol) and N,N-diisopropyl ethyl amine (13.6 mL, 78 mmol) in CH₂Cl₂ at 0° C. was added 2-bromo-5-methoxybenzoyl chloride (12.96 g, 52 mmol). The mixture was allowed to warm to room temperature and stirred for 2 h then poured into water and extracted with CH₂Cl₂. The organic layer was dried over anhydrous Na₂SO₄ and concentrated under reduced pressure. The crude material was purified by column chromatography (Petroleum ether/Ethyl acetate 70:30) to give compound (16 g, 85%). R_{f}. 0.25 (Petroleum ether/Ethyl acetate 70:30).

Further, trifluoroacetic acid (15 mL) was added slowly at 0-5° C. under nitrogen to a solution of tert-butyl-4-(2-bromo-5-methoxybenzoyl)piperazine-1-carboxylate (16 g, 40 mmol) in CH₂Cl₂ (15 mL). The mixture was stirred at room temperature for 2 h then poured on to cold water (50 mL). The aqueous solution was made basic by the addition of NaOH (1N), then the product was extracted into CH₂Cl₂ (2×100 mL). The combined extracts were washed with brine (200 mL), dried over anhydrous Na₂SO₄ and concentrated under reduced pressure yielding to 10.2 g (85%) of the title compound.

### Preparation of 4N-(2,4-difluorophenylsulfonyl)piperazine

To a solution of piperazine-1-carboxylic acid tert butyl ester (4.37 g, 23.5 mmol) and N,N-diisopropyl ethyl amine (6.14 mL, 35.3 mmol) in CH₂Cl₂ at 0° C. was added 2,4-difluorophenylsulfonyl chloride (5 g, 23.5 mmol). The mixture was allowed to warm to room temperature and stirred for 2 h then poured into water and extracted with CH₂Cl₂. The crude material was used in the next step without any purification. The solvent was dried over Na₂SO₄ and concentrated to dryness yielding to 8 g of the title compound (94%). Then the product, sudden a remplacement of the boc grouping according to higher described protocol, we so obtain a new reagent of the general synthesis.

### Example 1

### 1N-[(2-bromo-5-methoxybenzoyl)]-4N-[(phenyl)sulfonyl]piperazine

The general synthetic method described above affords as white solid from 1N-(2-bromo-5-methoxybenzoyl)piperazine (0.5 g, 1.67 mmol), and phenylsulfonyl chloride (0.21 mL, 1.67 mmol) and N,N-diisopropyl ethyl amine (0.44 mL, 2.51 mmol) in 20 mL of CH₂Cl₂. The crude product was crystallized from acetone to give pure product (0.47 g, 64%).

mp: 180° C.; ¹H NMR (300.13 MHz, CDCl3): δ 2.75 (m, 1H), 2.9 (m, 3H), 3.05 (m, 1H), 3.15 (m, 1H), 3.55 (s, OCH₃), 3.6 (m, 1H), 3.75 (m, 1H), 6.4 (d, J=3 Hz, 1H), 6.55 (dd, J=8.8 Hz, 3 Hz, 1H), 7.15 (dd, J=8.8 Hz, 1H), 7.3-7.5 (m, 3H), 7.65 (d, J=4.96 Hz, 2H); ¹³C NMR (75.46 MHz, CDCl3): δ 38.8, 43.5, 43.9, 43.9, 53.5, 106.9, 110.9, 114.5, 125.5 (2C), 127.1 (2C), 131.1, 131.6, 133.2, 135.6, 157, 165.2; HRMS (ESI) calc for [M+H]⁺ C₁₈H₂₀N₂O₄SBr 439.0327, obsd 439.0349; HPLC purity 94.34%, R_{T} 13.35.

### Example 2

### 1-[(2-bromo-5-methoxybenzoyl)]-4-[(4-fluorophenyl)sulfonyl]piperazine

The general synthetic method described above affords as white solid from 1N-(2-bromo-5-methoxybenzoyl)piperazine (0.5 g, 1.67 mmol), and 4-fluorophenylsulfonylchloride (0.32 g, 1.67 mmol) and N,N-diisopropyl ethyl amine (0.44 mL, 2.51 mmol) in 20 mL of CH₂Cl₂. The crude product was purified by column chromatography (Cyclohexane/Ethyl acetate 60:40) to give compound (0.52 g, 68%). R_{f}=0.33 (Cyclohexane/Ethyl acetate 60:40).

mp: 202° C.; ¹H NMR (300.13 MHz, CDCl3): δ 2.8-3.1 (m, 4H), 3.15-3.35 (m, 2H), 3.75 (m, 4H), 3.95 (m, 1H), 6.6 (d, J=3 Hz, 1H), 6.7 (dd, J=8.84 Hz, 3.02 Hz, 1H), 7.2 (m, 2H), 7.3 (d, J=8.85 Hz, 1H), 7.7 (m, 2H); ¹³C NMR (75.46 MHz, CDCl3): δ 40, 44.7, 45.1, 45.2, 54.7, 108.1, 112.2, 115.3, 115.7 (2C, J=10 Hz), 129.5 (2C, J=9 Hz), 130.6 (J=130 Hz), 132.8, 136.8, 158.3, 162.8, 164.5 (CF, J=255 Hz); ¹⁹F NMR (282.37 MHz, CDCl3) 6-103.9 (m, 1F); HRMS (ESI) calc for [M+H⁺] C₁₈H₁₉N₂O₄SBrF 457.0233, obsd 457.0213; HPLC purity 100%, R_{T} 13.76 min.

### Example 3

### 1N-[(2-bromo-5-methoxybenzoyl)]-4N-[(4-trifluoromethylphenyl)sulfonyl]piperazine

The general synthetic method described above affords as white solid from 1N-(2-bromo-5-methoxybenzoyl)piperazine (0.5 g, 1.67 mmol), and 4-trifluoromethylphenylsulfonylchloride (0.42 g, 1.67 mmol) and N,N-diisopropyl ethyl amine (0.44 mL, 2.51 mmol) in 20 mL of CH₂Cl₂. The crude product was purified by column chromatography (Cyclohexane/Ethyl acetate 50:50) to give compound (0.6 g, 71%). R_{f}=0.49 (Cyclohexane/Ethyl acetate 50:50).

mp: 155° C.; ¹H NMR (300.13 MHz, CDCl3): δ 2.90 (m, 1H), 3.05-3.15 (m, 3H), 3.2-3.4 (m, 2H), 3.7 (s, 3H, OCH₃), 3.75 (m, 1H), 3.9 (m, 1H) 6.65 (d, J=3 Hz, 1H), 6.7 (dd, J=8.8 Hz, 3 Hz, 1H), 7.35 (d, J=8.8 Hz, 1H), 7.7-7.9 (m, 4H); ¹³C NMR (75.46 MHz, CDCl3): δ 38.4, 43.1, 43.5, 43.6, 53.2, 106.5, 110.7, 114.2, 120.62 (q, CF₃, J=273.2 Hz), 124 (2C, J=3.8 Hz), 125.7 (2C), 131.3, 132.4 (C—CF₃, q, J=33 Hz), 135.1, 136.9, 156.7, 165; HRMS (ESI) calc for [M+H⁺] C₁₉H₁₉N₂O₄SBrF₃ 507.0201, obsd 507.0184; HPLC purity 100%, R_{T} 15.29 min.

### Example 4

### 1N-[(2-bromo-5-methoxybenzoyl)]-4N-[(4-acetylphenyl)sulfonyl]piperazine

The general synthetic method described above affords as white solid from 1N-(2-bromo-5-methoxybenzoyl)piperazine (0.5 g, 1.67 mmol), and 4-acetylphenylsulfonylchloride (0.36 g, 1.67 mmol) and N,N-diisopropyl ethyl amine (0.44 mL, 2.51 mmol) in 20 mL of CH₂Cl₂. The crude product was purified by column chromatography (Cyclohexane/Ethyl acetate 50:50) to give compound (0.55 g, 68%). R_{f}=0.15 (Cyclohexane/Ethyl acetate 50:50).

mp: 143° C.; ¹H NMR (300.13 MHz, CDCl3): δ 2.6 (s, 3H), 2.95 (m, 1H), 3-3.15 (m, 3H), 3.20-3.35 (m, 2H), 3.6 (s, 3H, OCH₃), 3.7 (m, 1H), 3.9 (m, 1H), 6.6 (d, J=3 Hz, 1H), 6.7 (dd, J=8.8 Hz, 3.03 Hz, 1H), 7.3 (d, J=8.8 Hz, 1H), 7.8 (d, J=6.8 Hz, 1.7 Hz, 2H), 8.05 (dd, J=6.8 Hz, 1.7 Hz, 2H); ¹³C NMR (75.46 MHz, CDCl3): δ 24.4, 38.4, 43.1, 43.5, 43.6, 53.2, 106.6, 110.7, 114.2, 125.5 (2C), 126.6 (2C), 131.3, 135.2, 137.1, 138, 156.7, 165, 194.1; HRMS (ESI) calc for [M+H⁺] C₂₀H₂₂N₂O₅SBr 481.0433, obsd 481.0437; HPLC purity 95.5%, R_{T} 13.17 min.

### Example 5

### 1N-[(2-bromo-5-methoxybenzoyl)]-4N-[(4-propylphenyl)sulfonyl]piperazine

The general synthetic method described above affords as white solid from 1N-(2-bromo-5-methoxybenzoyl)piperazine (0.5 g, 1.67 mmol), and 4-propylphenylsulfonylchloride (0.3 mL, 1.67 mmol) and N,N-diisopropyl ethyl amine (0.44 mL, 2.51 mmol) in 20 mL of CH₂Cl₂. The crude product was purified by column chromatography (Petroleum ether/Ethyl acetate 50:50) to give compound (0.62 g, 77%). R_{f}=0.45 (Petroleum ether/Ethyl acetate 50:50).

mp: 134° C.; ¹H NMR (300.13 MHz, CDCl3): δ 0.88 (t, J=7.3 Hz, 3H), 1.61 (m, 2H), 2.61 (t, J=7.6 Hz, 2H), 2.86-3.1 (m, 4H), 3.17-3.36 (m, 2H), 3.67-3.75 (m, 1H), 3.69 (s, 3H, OCH₃), 3.87-3.95 (m, 1H), 6.6 (d, J=3 Hz, 1H), 6.7 (dd, J=8.8 Hz, 3 Hz, 1H), 7.25-7.35 (m, 3H), 7.65 (d, J=8.35 Hz, 2H); ¹³C NMR (75.46 MHz, CDCl3): δ 15.3, 25.8, 39.5, 42.6, 47.3, 47.7, 47.8, 57.3, 110.7, 114.8, 118.3, 129.4 (2C), 130.9 (2C), 134.1, 135.4, 139.4, 150.4, 160.8, 169.1; HRMS (ESI) calc for [M+H]⁺ C₂₁H₂₆N₂O₄SBr 481.0797, obsd 481.0777; HPLC purity 100%, R_{T} 15.95 min.

### Example 6

### 1N-[(2-bromo-5-methoxybenzoyl)]-4N-[(4-nitrophenyl)sulfonyl]piperazine

The general synthetic method described above affords as white solid from 1N-(2-bromo-5-methoxybenzoyl)piperazine (0.5 g, 1.67 mmol), and 4-nitrophenylsulfonylchloride (0.37 g, 1.67 mmol) and N,N-diisopropyl ethyl amine (0.44 mL, 2.51 mmol) in 20 mL of CH₂Cl₂. The crude product was purified by column chromatography (Petroleum ether/Ethyl acetate 50:50) to give compound (0.62 g, 77%). R_{f}=0.37 (Petroleum ether/Ethyl acetate 50:50).

mp: 151° C.; ¹H NMR (300.13 MHz, CDCl3): δ 2.9-3.35 (m, 6H), 3.65 (s, 3H, OCH₃), 3.7 (m, 1H), 3.85 (m, 1H), 6.6 (d, J=3 Hz, 1H), 6.75 (dd, J=8.9 Hz, 3 Hz, 1H), 7.35 (d, J=8.8 Hz, 1H), 7.85 (d, J=2.3 Hz, 2H), 8.35 (d, J=2.3 Hz, 2H); ¹³C NMR (75.46 MHz, CDCl3): δ 40.9, 45.6, 46, 46.1, 55.7, 109, 113.2, 116.7, 124.5 (2C), 128.8 (2C), 133.8, 137.5, 141.8, 150.4, 159.2, 167.4; HRMS (ESI) calc for [M+H]⁺ C₁₈H₁₉N₃O₆SBr 484.0178, obsd 484.0186; HPLC purity 97%, R_{T} 14 min.

### Example 7

### 1N-[(2-bromo-5-methoxybenzoyl)]-4N-[(3-trifluoromethylphenyl)sulfonyl]piperazine

The general synthetic method described above affords as white solid from 1N-(2-bromo-5-methoxybenzoyl)piperazine (0.5 g, 1.67 mmol), and 3-trifluoromethylphenylsulfonylchloride (0.28 mL, 1.67 mmol) and N,N-diisopropyl ethyl amine (0.44 mL, 2.51 mmol) in 20 mL of CH₂Cl₂. The crude product was purified by column chromatography (Cyclohexane/Ethyl acetate 50:50) to give compound (0.69 g, 81%). R_{f}=0.44 (Cyclohexane/Ethyl acetate 50:50).

mp: 154° C.; ¹H NMR (300.13 MHz, CDCl3): δ 2.90 (m, 1H), 3.05-3.15 (m, 3H), 3.2-3.4 (m, 2H), 3.7 (s, 3H, OCH₃), 3.75 (m, 1H), 3.9 (m, 1H) 6.65 (d, J=3 Hz, 1H), 6.7 (dd, J=8.8 Hz, 3 Hz, 1H), 7.35 (d, J=8.8 Hz, 1H), 7.65 (t, J=7.8 Hz, 1H), 7.8-7.95 (m, 3H); ¹³C NMR (75.46 MHz, CDCl3): δ 40.9, 45.6, 46, 46.1, 55.6, 109, 113.1, 116.8, 123.1 (CF₃, J=273.2 Hz), 124.6 (J=3.8 Hz), 129.9 (J=3.8 Hz), 130.2, 130.8, 132.1 (C—CF₃, J=34 Hz), 133.8, 137, 137.6, 159.2, 167.5; HRMS (ESI) calc for [M+H⁺] C₁₉H₁₉N₂O₄SBrF₃ 507.0201, obsd 507.0182; HPLC purity 100%, R_{T} 15.15 min.

### Example 8

### 1N-[(2-bromo-5-methoxybenzoyl)]-4N-[(2-trifluoromethylphenyl)sulfonyl]piperazine

The general synthetic method described above affords as white solid from 1N-(2-bromo-5-methoxybenzoyl)piperazine (0.5 g, 1.67 mmol), and 2-trifluoromethylphenylsulfonylchloride (0.26 mL, 1.67 mmol) and N,N-diisopropyl ethyl amine (0.44 mL, 2.51 mmol) in 20 mL of CH₂Cl₂. The crude product was purified by column chromatography (Cyclohexane/Ethyl acetate 50:50) to give compound (0.51 g, 60%). R_{f}=0.37 (Cyclohexane/Ethyl acetate 50:50).

mp: 136.5° C.; ¹H NMR (300.13 MHz, CDCl3): δ 3.15-3.45 (m, 6H), 3.75 (m, 4H), 4.05 (m, 1H), 6.75 (d, J=3 Hz, 1H), 6.8 (dd, J=8.8 Hz, 3 Hz, 1H), 7.45 (d, J=8.8 Hz, 1H), 7.7 (m, 2H), 7.85 (m, 1H), 8.05 (m, 1H); ¹³C NMR (75.46 MHz, CDCl3): δ 39.7, 43.7, 44, 45, 54.1, 107.6, 111.6, 115.3, 120.9 (CF₃, J=274 Hz), 126.6 (C—CF₃, J=33 Hz), 127.2 (CH, J=6.8 Hz), 130.6, 130.8, 131.6, 132.2, 135.7, 136.3, 157.7, 166; HRMS (ESI) calc for [M+H⁺] C₁₉H₁₉N₂O₄SBrF₃ 507.0201, obsd 507.0185; HPLC purity 98.5%, R_{T} 14.67 min.

### Example 9

### 1N-[(2-bromo-5-methoxybenzoyl)]-4N-[(4-biphenyl)sulfonyl]piperazine

The general synthetic method described above affords as white solid from 1N-(2-bromo-5-methoxybenzoyl)piperazine (0.5 g, 1.67 mmol), and 4-biphenylsulfonylchloride (0.42 g, 1.67 mmol) and N,N-diisopropyl ethyl amine (0.44 mL, 2.51 mmol) in 20 mL of CH₂Cl₂. The crude product was purified by column chromatography (Cyclohexane/Ethyl acetate 50:50) to give compound 0.62 g, 72%). R_{f}=0.47 (Cyclohexane/Ethyl acetate 50:50).

mp: 100° C.; ¹H NMR (300.13 MHz, CDCl3): δ 2.95 (m, 1H), 3-3.15 (m, 3H), 3.20-3.40 (m, 2H), 3.6 (s, 3H, OCH₃), 3.7 (m, 1H), 3.85 (m, 1H), 6.6 (d, J=3 Hz, 1H), 6.75 (dd, J=8.8 Hz, 3 Hz, 1H), 7.3-7.45 (m, 4H), 7.55 (d, 2H), 7.7 (m, 4H); ¹³C NMR (75.46 MHz, CDCl3): δ 41, 45.7, 46.1, 46.2, 55.6, 109.1, 113.1, 116.7, 127.34, 127.8 (2C), 128.2 (2C), 128.7 (2C), 129.1 (2C), 133.7, 133.8, 137.7, 139, 146.2, 159.2, 167.5; HRMS (ESI) calc for [M+H⁺] C₂₄H₂₄N₂O₄SBr 515.0640, obsd 515.0628; HPLC purity 97%, R_{T} 16.05 min.

### Example 10

### 1N-[(2-bromo-5-methoxybenzoyl)]-4N-[(4-methylphenyl)sulfonyl]piperazine

The general synthetic method described above affords as white solid from 1N-(2-bromo-5-methoxybenzoyl)piperazine (0.5 g, 1.67 mmol), and tosylchloride (0.32 g, 1.67 mmol) and N,N-diisopropyl ethyl amine (0.44 mL, 2.51 mmol) in 20 mL of CH₂Cl₂. The crude product was purified by column chromatography (Petroleum ether/Ethyl acetate 50:50) to give compound (0.51 g, 67%). R_{f}=0.39 (Petroleum ether/Ethyl acetate 50:50).

mp: 178° C.; ¹H NMR (300.13 MHz, CDCl3): δ 2.35 (s, 3H), 2.85 (m, 1H), 3.00 (m, 3H), 3.15 (m, 1H), 3.25 (m, 1H), 3.65 (s, OCH₃), 3.75 (m, 1H), 3.85 (m, 1H), 6.55 (d, J=3 Hz, 1H), 6.70 (dd, J=8.8 Hz, 3 Hz, 1H), 7.25-7.4 (m, 3H), 7.6 (d, J=8.2 Hz, 2H); ¹³C NMR (75.46 MHz, CDCl3): δ 19.6, 38.9, 43.7, 44.1, 44.1, 53.65, 107.1, 111.2, 114.6, 125.7 (2C), 127.9 (2C), 130.4, 131.7, 135.8, 142.2, 157.2, 165.4; HRMS (ESI) calc for [M+H]⁺ C₁₉H₂₂N₂O₄SBr 453.0483, obsd 453.0484; HPLC purity 99.6%, R_{T} 14.16 min.

### Example 11

### [(2-bromo-5-methoxybenzoyl)]-4N-(4-trifluoromethoxyphenyl)sulfonyl]piperazine

The general synthetic method described above affords as white solid from 1N-(2-bromo-5-methoxybenzoyl)piperazine (0.5 g, 1.67 mmol), and 4-trifluoromethoxyphenylsulfonylchloride (0.29 mL, 1.67 mmol) and N,N-diisopropyl ethyl amine (0.44 mL, 2.51 mmol) in 20 mL of CH₂Cl₂. The crude product was crystallized from acetone to give pure product (0.6 g, 69%).

mp: 110.4° C.; ¹H NMR (300.13 MHz, CDCl3): δ 2.90 (m, 1H), 2.95-3.15 (m, 3H), 3.2-3.4 (m, 2H), 3.7 (s, 3H, OCH₃), 3.75 (m, 1H), 3.9 (m, 1H) 6.65 (d, J=3 Hz, 1 H), 6.7 (dd, J=8.8 Hz, 3 Hz, 1H), 7.25-7.4 (m, 3H), 7.75 (m, 2H); ¹³C NMR (75.46 MHz, CDCl3): δ 40.9, 45.6, 46, 46.1, 55.6, 109, 113.1, 116.7, 118.47, 121.1 (2C), 129.8 (2C), 133.79, 133.9, 137.7, 152.6, 159.2, 167.5; ¹⁹F NMR (282.37 MHz, CDCl3) 6-57.6 (s, 3F); HRMS (ESI) calc for [M+H⁺] C₁₉H₁₉N₂O₅SBrF₃ 523.0150, obsd 523.0130; HPLC purity 100%, R_{T} 15.43 min.

### Example 12

### 1N-[(2-bromo-5-methoxybenzoyl)]-4N-[(2, 4 difluorophenyl)sulfonyl]piperazine

The general synthetic method described above affords as white solid from 1N-(2-bromo-5-methoxybenzoyl)piperazine (0.5 g, 1.67 mmol), and 2,4-difluorophenylsulfonylchloride (0.22 mL, 1.67 mmol) and N,N-diisopropyl ethyl amine (0.44 mL, 2.51 mmol) in 20 mL of CH₂Cl₂. The crude product was crystallized from acetone to give pure product (0.6 g, 75%).

mp: 194° C.; ¹H NMR (300.13 MHz, CDCl3): δ 3.15-3.45 (m, 6H), 3.75 (m, 4H), 4.05 (m, 1H), 6.75 (d, J=3 Hz, 1H), 6.8 (dd, J=8.8 Hz, 3 Hz, 1H), 7.05 (m, 2H), 7.45 (d, J=8.8 Hz, 1H), 7.90 (m, 1H); ¹³C NMR (75.46 MHz, CDCl3): δ 41.2, 45.4, 45.8, 46.4, 55.6, 105.6 (J=25.7 Hz), 109.1, 112.2 (J=3.8 Hz), 113.2, 116.7, 121.7 (J=3.8 Hz), 132.9 (J=10.6 Hz), 133.8, 137.8, 159.2, 159.7 (J=261.1 Hz), 162.8 (J=258.8 Hz), 167.6; ¹⁹F NMR (282.4 MHz, CDCl3) 6-102.3 (m, 1F), −99.5 (m, 1F); HRMS (ESI) calc for [M+H'] C₁₈H₁₈N₂O₄SBrF₂475.0139, obsd 475.0143; HPLC purity 97.9%, R_{T} 13.98 min.

### Example 13

### 1N-[(2-bromo-5-methoxybenzoyl)]-4N-[(2-fluorophenyl)sulfonyl]piperazine

The general synthetic method described above affords as white solid from 1-(2-bromo-5-methoxybenzoyl)piperazine (0.5 g, 1.67 mmol), and 2-fluorophenylsulfonylchloride (0.22 mL, 1.67 mmol) and N,N-diisopropyl ethyl amine (0.44 mL, 2.51 mmol) in 20 mL of CH₂Cl₂. The crude product was crystallized from acetone to give pure product (0.52 g, 68%).

mp: 167.3° C.; ¹H NMR (300.13 MHz, CDCl3): δ 3.10 (m, 1H), 3.15-3.4 (m, 5H), 3.7 (s, 3H, OCH₃), 3.75 (m, 1H), 3.9 (m, 1H) 6.65 (d, J=3 Hz, 1H), 6.7 (dd, J=8.8 Hz, 3 Hz, 1H), 7.15-7.25 (m, 2H), 7.3 (d, J=8.8 Hz, 1H), 7.5 (m, 1H), 7.75 (m, 1H); ¹³C NMR (75.46 MHz, CDCl3): δ 43, 47.1, 47.5, 48.2, 57.3, 110.8, 114.9, 118.4, 119.1 (J=21 Hz), 126.4 (J=3 Hz), 126.8 (J=14 Hz), 132.9, 135.5, 137.2 (J=8 Hz), 139.5, 160.6 (CF, J=255 Hz), 160.9, 169.2; HRMS (ESI) calc for [M+H⁺] C₁₈H₁₉N₂O₄SbrF 457.0227, obsd 457.0233; HPLC purity 100%, R_{T} 13.57 min.

### Example 14

### 1N-[(2-bromo-5-methoxybenzoyl)]-4N-[(2,6-difluorophenyl)sulfonyl]piperazine

The general synthetic method described above affords as white solid from 1N-(2-bromo-5-methoxybenzoyl)piperazine (0.5 g, 1.67 mmol), and 2,6 difluorophenylsulfonylchloride (0.36 g, 1.67 mmol) and N,N-diisopropyl ethyl amine (0.44 mL, 2.51 mmol) in 20 mL of CH₂Cl₂. The crude product was crystallized from acetone to give pure product (0.55 g, 69%).

mp: 154.5° C.; ¹H NMR (300.13 MHz, CDCl3): δ 3.15-3.45 (m, 6H), 3.75 (m, 4H), 3.95 (m, 1H) 6.65 (d, J=3 Hz, 1H), 6.7 (dd, J=8.8 Hz, 3 Hz, 1H), 7.05 (t, J=8.7 Hz, 2H), 7.45 (d, J=8.8 Hz, 1H), 7.5 (m, 1H); ¹³C NMR (75.46 MHz, CDCl3): δ 41.2, 45.3, 45.7, 46.4, 55.6, 109.1, 113.1, 113.5 (2C, J=3.8 Hz), 115.2 (J=16.6 Hz), 116.7, 133.8, 135.1, 137.7, 159.2, 159.7 (J=259 Hz, 2C—F), 167.5; ¹⁹F NMR (282.4 MHz, CDCl3) δ-(m, 1F), -(m, 1F); HRMS (ESI) calc for [M+H⁺] C₁₈H₁₈N₂O₄SBrF₂ 475.0139 obsd 475.0143; HPLC purity 92.4%, R_{T} 13.65 min.

### Example 15

### 1N-[(2-bromo-5-methoxybenzoyl)]-4N-[(3-fluorophenyl)sulfonyl]piperazine

The general synthetic method described above affords as white solid from 1N-(2-bromo-5-methoxybenzoyl)piperazine (0.5 g, 1.67 mmol), and 3-fluorophenylsulfonylchloride (0.23 mL, 1.67 mmol) and N,N-diisopropyl ethyl amine (0.44 mL, 2.51 mmol) in 20 mL of CH₂Cl₂. The crude product was crystallized from acetone to give pure product (0.56 g, 73%).

mp: 145° C.; ¹H NMR (300.13 MHz, CDCl3): δ 2.93 (m, 1H), 3-3.15 (m, 3H), 3.20-3.40 (m, 2H), 3.7 (s, 3H, OCH₃), 3.75 (m, 1H), 3.9 (m, 1H) 6.6 (d, J=3 Hz, 1H), 6.7 (dd, J=8.8 Hz, 3 Hz, 1H), 7.2-7.4 (m, 3H), 7.5 (m, 2H); ¹³C NMR (75.46 MHz, CDCl3): δ 40.9, 45.7, 46, 46.1, 55.6, 109.1, 113.2, 115 (J=24 Hz), 116.7, 120.5 (J=21 Hz), 123.4 (J=3.8 Hz), 131.1 (J=7.5 Hz), 133.8, 137.6 (J=6 Hz), 137.7, 159.2, 162.5 (J=252 Hz), 167.5; HRMS (ESI) calc for [M+H⁺] C₁₈H₁₉N₂O₄SBrF 457.0233, obsd 457.0237; HPLC purity 98.9%, R_{T} 13.89 min.

### Example 16

### 1N-[(2-bromo-5-methoxybenzoyl)]-4N-[(4-isopropylphenyl)sulfonyl]piperazine

The general synthetic method described above affords as white solid from 1N-(2-bromo-5-methoxybenzoyl)piperazine (0.5 g, 1.67 mmol), and 4-isopropylphenylsulfonylchloride (0.3 mL, 1.67 mmol) and N,N-diisopropyl ethyl amine (0.44 mL, 2.51 mmol) in 20 mL of CH₂Cl₂. The crude product was purified by column chromatography (Petroleum ether/Ethyl acetate 50:50) to give compound (0.55 g, 68%). R_{f}=0.52 (Petroleum ether/Ethyl acetate 50:50).

mp: 123° C.; ¹H NMR (300.13 MHz, CDCl3): δ 1.25 (s, 6H), 2.9-3.15 (m, 5H), 3.2-3.4 (m, 2H), 3.65 (s, 3H, OCH₃), 3.7 (m, 1H), 3.85 (m, 1H), 6.6 (d, J=3 Hz, 1H), 6.7 (dd, J=8.8 Hz, 3 Hz, 1H), 7.4 (m, 3H), 7.65 (d, J=8.4 Hz, 2H); ¹³C NMR (75.46 MHz, CDCl3): δ 23.6 (2 C), 34.2, 41, 45.6, 46.1, 46.1, 55.6, 109.1, 113.1, 116.6, 127.3 (2C), 127.9 (2C), 132.5, 133.7, 137.8, 154.8, 159.2, 167.4; HRMS (ESI) calc for [M+H]⁺ C₂₁H₂₆N₂O₄SBr 481.0797, obsd 481.0791; HPLC purity 99.6%, R_{T} 15.77 min.

### Example 17

### 1N-[(2-bromo-5-methoxybenzoyl)]-4N-[(4-bromophenyl)sulfonyl]piperazine

The general synthetic method described above affords as white solid from 1N-(2-bromo-5-methoxybenzoyl)piperazine (0.5 g, 1.67 mmol), and 4-bromophenylsulfonylchloride (0.43 g, 1.67 mmol) and N,N-diisopropyl ethyl amine (0.44 mL, 2.51 mmol) in 20 mL of CH₂Cl₂. The crude product was crystallized from acetone to give pure product (0.56 g, 65%).

mp: 180° C.; ¹H NMR (300.13 MHz, CDCl3): δ 2.9 (m, 1H), 3.05 (m, 3H), 3.15-3.35 (m, 2H), 3.65 (s, 3H, OCH₃), 3.7 (m, 1H), 3.85 (m, 1H), 6.6 (d, J=3 Hz, 1H), 6.75 (dd, J=8.9 Hz, 3 Hz, 1H), 7.3 (d, 1H), 7.5 (d, J=2.3 Hz, 2H), 7.65 (d, J=2.3 Hz, 2H); ¹³C NMR (75.46 MHz, CDCl3): δ 40.9, 45.6, 46, 46.1, 55.7, 109.1, 113.2, 116.7, 128.4, 129.1 (2C), 132.6 (2C), 133.7, 134.6, 137.7, 159.2, 167.4; HRMS (ESI) calc for [M+H]⁺ C₁₈H₁₉N₂O₄SBr₂ 516.9432, obsd 516.9438; HPLC purity 98.2%, R_{T} 14.93 min.

### Example 18

### 1N-[(2-bromo-5-methoxybenzoyl)]-4N-[(4-chlorophenyl)sulfonyl]piperazine

The general synthetic method described above affords as white solid from 1N-(2-bromo-5-methoxybenzoyl)piperazine (0.5 g, 1.67 mmol), and 4-chlorophenylsulfonylchloride (0.36 g, 1.67 mmol) and N,N-diisopropyl ethyl amine (0.44 mL, 2.51 mmol) in 20 mL of CH₂Cl₂. The crude product was crystallized from acetone to give pure product (0.5 g, 63%).

mp: 188° C.; ¹H NMR (300.13 MHz, CDCl3): δ 2.9 (m, 1H), 2.95-3.05 (m, 3H), 3.15-3.35 (m, 2H), 3.65 (s, 3H, OCH₃), 3.7 (m, 1H), 3.85 (m, 1H), 6.6 (d, J=3 Hz, 1H), 6.7 (dd, J=8.8 Hz, 3 Hz, 1H), 7.35 (d, J=8.8 Hz, 1H), 7.45 (d, J=Hz, 2H), 7.6 (d, J=Hz, 2H); ¹³C NMR (75.46 MHz, CDCl3): δ 40.9, 45.6, 46, 46.1, 55.6, 109.1, 113.2, 116.7, 129.1 (2C), 129.6 (2C), 133.7, 134.1, 137.7, 139.9, 159.2, 167.4; HRMS (ESI) calc for [M+H]⁺ C₁₈H₁₉N₂O₄SBrC1472.9914, obsd 472.9937; HPLC purity 100%, R_{T} 14.60 min.

### Example 19

### 1N-[(2-bromo-5-methoxybenzoyl)]-4N-[(4-methoxyphenyl)sulfonyl]piperazine

The general synthetic method described above affords as white solid from 1N-(2-bromo-5-methoxybenzoyl)piperazine (0.5 g, 1.67 mmol), and 4-methoxyphenylsulfonylchloride (0.34 g, 1.67 mmol) and N,N-diisopropyl ethyl amine (0.44 mL, 2.51 mmol) in 20 mL of CH₂Cl₂. The crude product was purified by column chromatography (Cyclohexane/Ethyl acetate 50:50) to give compound (0.52 g, 66%). R_{f}=0.296 (Cyclohexane/Ethyl acetate 50:50).

mp: 187° C.; ¹H NMR (300.13 MHz, CDCl3): δ 2.9 (m, 1H), 3.05 (m, 3H), 3.15-3.35 (m, 2H), 3.65 (s, OCH₃), 3.7 (m, 1H), 3.75 (s, 3H, OCH₃), 3.8 (m, 1H), 6.6 (d, J=3 Hz, 1H), 6.7 (dd, J=8.8 Hz, 3 Hz, 1H), 6.9 (m, 2H), 7.35 (d, J=8.8 Hz, 1H), 7.65 (m, 2H); ¹³C NMR (75.46 MHz, CDCl3): δ 40.6, 45.4, 45.8, 45.8, 55.36, 55.40, 108.8, 112.9, 114.1 (2C), 116.4, 126.6, 129.6 (2C), 133.5, 137.5, 158.9, 163.1, 167.2; HRMS (ESI) calc for [M+H⁺] C₁₉H₂₂N₂O₅SBr 469.0428, obsd 469.0433; HPLC purity 100%, R_{T} 13.66 min.

### Example 20

### 1N-[(2-bromo-5-methoxybenzoyl)]-4N-[(phenylmethane)sulfonyl]piperazine

The general synthetic method described above affords as white solid from 1N-(2-bromo-5-methoxybenzoyl)piperazine (0.5 g, 1.67 mmol), and phenylmethanesulfonylchloride (0.32 g, 1.67 mmol) and N,N-diisopropyl ethyl amine (0.44 mL, 2.51 mmol) in 20 mL of CH₂Cl₂. The crude product was purified by column chromatography (Cyclohexane/Ethyl acetate 40:60) to give compound (0.5 g, 66%). R_{f}=0.32 (Cyclohexane/Ethyl acetate 50:50).

mp: 112° C.; ¹H NMR (300.13 MHz, CDCl3): δ 2.95 (m, 4H), 3.1-3.3 (m, 2H), 3.45 (m, 1H), 3.65 (s, 3H, OCH₃), 3.7 (s, 3H), 3.9 (m, 1H), 4.2 (s, 2H), 6.6 (d, J=3 Hz, 1H), 6.7 (dd, J=8.8 Hz, 3 Hz, 1H), 7.3 (m, 6H); ¹³C NMR (75.46 MHz, CDCl3): δ 41.9, 45.9, 46.4, 47.2, 55.9, 57.8, 109.3, 113.3, 117, 128.6, 129.2 (2C), 129.3, 130.9 (2C), 134, 138.1, 159.5, 167.7; HRMS (ESI) calc for [M+H⁺] C₁₉H₂₂N₂O₄SBr 453.0484, obsd 453.0483; HPLC purity 99%, R_{T} 13.54 min.

### Example 21

### 1N-[(2-bromo-5-methoxybenzoyl)]-4N-[(2-thiophene)sulfonyl]piperazine

The general synthetic method described above affords as white solid from 1N-(2-bromo-5-methoxybenzoyl)piperazine (0.25 g, 0.84 mmol), and 2-thiophenesulfonylchloride (0.158 g, 0.84 mmol) and N,N-diisopropyl ethyl amine (0.22 mL, 1.25 mmol) in 10 mL of CH₂Cl₂. The crude product was crystallized from acetone to give pure product (0.27 g, 72%).

mp: 155.8° C.; ¹H NMR (300.13 MHz, CDCl3): δ 2.95 (m, 1H), 3-3.2 (m, 4H), 3.2-3.4 (m, 1H), 3.75 (m, 4H), 3.95 (m, 1H), 6.65 (d, J=3 Hz, 1H), 6.7 (dd, J=8.8 Hz, 3 Hz, 1H), 7.05 (t, J=Hz, 1H), 7.35 (d, J=8.8 Hz, 1H), 7.45 (dd, J=3.7 Hz, 1.3 Hz, 1H), 7.6 (dd, J=5 Hz, 1.3 Hz, 1H); HRMS (ESI) calc for [M+H⁺] C₁₆H₁₈N₂O₄S₂Br 441.9891, obsd 441.9883; HPLC purity 99.5%, R_{T} 13.32 min.

### Example 22

### 1N-[(2-bromo-5-methoxybenzoyl)]-4N-[(3-thiophene)sulfonyl]piperazine

The general synthetic method described above affords as white solid from 1-(2-bromo-5-methoxybenzoyl)piperazine (0.25 g, 0.84 mmol), and 3 thiophenesulfonylchloride (0.158 g, 0.84 mmol) and N,N-diisopropyl ethyl amine (0.22 mL, 1.25 mmol) in 10 mL of CH₂Cl₂. The crude product was crystallized from acetone to give pure product (0.27 g, 67%).

mp: 156.9° C.; ¹H NMR (300.13 MHz, CDCl3): δ 2.90-3.35 (m, 6H), 3.75 (m, 4H), 3.95 (m, 1H), 6.60 (d, J=3 Hz, 1H), 6.7 (dd, J=8.8 Hz, 3 Hz, 1H), 7.2 (m, 1H), 7.35 (d, J=8.8 Hz, 1H), 7.45 (dd, J=5.1 Hz, 3.1 Hz, 1H), 7.85 (dd, J=3 Hz, 1.3 Hz, 1H); ¹³C NMR (75.46 MHz, CDCl3): δ 38.50, 43.19, 43.59, 43.69, 53.24, 106.67, 110.72, 114.29, 123.36, 125.81, 128.81, 131.36, 133.15, 135.34, 156.81, 165.05; HRMS (ESI) calc for [M+H⁺] C₁₆H₁₈N₂O₄S₂Br 441.9891, obsd 441.9884; HPLC purity 100%, R_{T} 12.92 min.

### Example 23

### 1N-[(2-bromo-5-methoxybenzoyl)]-4N-[(2-furan)sulfonyl]piperazine

The general synthetic method described above affords as white solid from 1-(2-bromo-5-methoxybenzoyl)piperazine (0.25 g, 0.84 mmol), and 2-furanesulfonylchloride (0.140 g, 0.84 mmol) and N,N-diisopropyl ethyl amine (0.22 mL, 1.25 mmol) in 10 mL of CH₂Cl₂. The crude product was crystallized from acetone to give pure product (0.23 g, 64%).

mp: 132.8° C.; ¹H NMR (300.13 MHz, CDCl3): δ 3.05-3.35 (m, 6H), 3.75 (m, 4H), 3.95 (m, 1H), 6.45 (dd, J=3.5 Hz, 1.8 Hz, 1H), 6.65 (d, J=3 Hz, 1H), 6.75 (dd, J=8.8 Hz, 3 Hz, 1H), 7 (dd, J=3.5 Hz, 0.8 Hz, 1H), 7.35 (d, J=8.8 Hz, 1H), 7.5 (dd, J=1.7 Hz, 0.8 Hz, 1H); ¹³C NMR (75.46 MHz, CDCl3): δ 40.95, 45.54, 45.94, 46.14, 55.66, 109.09, 111.38, 113.18, 116.70, 117.55, 133.79, 137.76, 146.45, 146.85, 159.23, 167.51; HRMS (ESI) calc for [M+H⁺] C₁₆H₁₈N₂O₅SBr 429.0120, obsd 429.0122; HPLC purity 100%, R_{T} 12.87 min.

### Example 24

### 1N-[(2-bromo-5-methoxybenzoyl)]-4N-[(3-furan)sulfonyl]piperazine

The general synthetic method described above affords as white solid from 1-(2-bromo-5-methoxybenzoyl)piperazine (0.25 g, 0.84 mmol), and 3-furanesulfonylchloride (0.140 g, 0.84 mmol) and N,N-diisopropyl ethyl amine (0.22 mL, 1.25 mmol) in 10 mL of CH₂Cl₂. The crude product was crystallized from acetone to give pure product (0.26 g, 72%).

mp: 162.3° C.; ¹H NMR (300.13 MHz, CDCl3): δ 2.9-3.4 (m, 6H), 3.75 (m, 4H), 3.95 (m, 1H), 6.45 (dd, J=1.9 Hz, 0.7 Hz, 1H), 6.65 (d, J=3 Hz, 1H), 6.75 (dd, J=8.8 Hz, 3 Hz, 1H), 7.35 (d, J=8.8 Hz, 1H), 7.45 (t, J=1.8 Hz, 1H), 7.85 (dd, J=1.4 Hz, 0.7 Hz, 1H); ¹³C NMR (75.46 MHz, CDCl3): δ 40.85, 45.51, 45.89, 46.05, 55.66, 108.6, 109.08, 113.17, 116.72, 122.74, 133.78, 137.75, 144.97, 146.06, 159.24, 167.47; HRMS (ESI) calc for [M+H]⁺ C₁₆H₁₈N₂O₅SBr 429.0120, obsd 429.0110; HPLC purity 100%, R_{T} 12.46 min.

### Example 25

### 1N-[(2-bromo-5-methoxybenzoyl)]-4N-[(1-methyl-1H-imidazole)sulfonyl]piperazine

The general synthetic method described above affords as white solid from 1-(2-bromo-5-methoxybenzoyl)piperazine (0.25 g, 0.84 mmol), and 1-methyl-1H-imidazolesulfonylchloride (0.151 g, 0.84 mmol) and N,N-diisopropyl ethyl amine (0.22 mL, 1.25 mmol) in 10 mL of CH₂Cl₂. The crude product was crystallized from acetone to give pure product (0.29 g, 78%).

mp: 190.2° C.; ¹H NMR (300.13 MHz, CDCl3): δ 3.1-3.4 (m, 6H), 3.75 (s, 6H), 3.95 (m, 2H), 6.65 (d, J=3 Hz, 1H), 6.75 (dd, J=8.8 Hz, 3 Hz, 1H), 7.35 (s, 1H), 7.38 (m, 1H), 7.43 (m, 1H); ¹³C NMR (75.46 MHz, CDCl3): δ 34.08, 40.95, 45.75, 46.25, 46.32, 55.65, 109.14, 113.20, 116.60, 124.80, 133.72, 137.68, 137.93, 139.36, 159.17, 167.49; HRMS (ESI) calc for [M+H⁺] C₁₆H₂₀N₄O₄SBr 443.0402, obsd 443.0389; HPLC purity.

### Example 26

### 1N-[(3-bromo-4-methoxybenzoyl)]-4-N-[(2,4-difluorophenyl)sulfonyl]piperazine

The general synthetic method described above affords as white solid from 4N-(2,4-difluorophenylsulfonyl)piperazine (0.27 g, 1.03 mmol), and 3-bromo-4-methoxybenzoyl chloride (0.26 g, 1.03 mmol) and N,N-diisopropyl ethyl amine (0.27 mL, 1.56 mmol) in 10 mL of CH₂Cl₂. The crude product was purified by column chromatography (Cyclohexane/Ethyl acetate 50:50) to give compound (0.31 g, 63%). R_{f}=0.33 (Cyclohexane/Ethyl acetate 50:50).

mp: 166.7° C.; ¹H NMR (300.13 MHz, CDCl3): δ 3.15 (m, 4H), 3.75 (m, 4H), 3.85 (s, 3H), 6.7 (d, J=8.8 Hz, 1H), 6.9 (m, 2H), 7.25 (d, 2.5 Hz, 1H), 7.4 (dd, J=8.8 Hz, 2.5 Hz, 1H), 7.8 (m, 1H); ¹³C NMR (75.46 MHz, CDCl3): δ 45.73, 56.40, 106.01 (J=25.7 Hz), 111.47, 111.84, 112.25 (J=3.8 Hz), 121.4 (J=3.8 Hz), 127.99, 128.15, 132.91 (J=9.8 Hz), 157.47, 159.78 (J=258.8 Hz), 166 (J=258.8 Hz), 168.98; HRMS (ESI) calc for [M+H⁺] C₁₈H₁₈N₂O₄SBrF₂ 475.0139, obsd 475.0152; HPLC purity 97.7%, R_{T} 14.11 min.

### Example 27

### 1N-[(2-methoxy-5-bromobenzoyl)]-4-N-[(2,4-difluorophenyl)sulfonyl]piperazine

The general synthetic method described above affords as white solid from 4N-(2,4-difluorophenylsulfonyl)piperazine (0.26 g, 0.99 mmol), and 2-methoxy-5-bromobenzoyl chloride (0.25 g, 0.99 mmol) and N,N-diisopropyl ethyl amine (0.26 mL, 1.49 mmol) in 10 mL of CH₂Cl₂. The crude product was purified by column chromatography (Cyclohexane/Ethyl acetate 50:50) to give compound (0.32 g, 68%). R_{f}=0.49 (Cyclohexane/Ethyl acetate 50:50).

mp: 171.2° C.; ¹H NMR (300.13 MHz, CDCl3): δ 2.95-3.4 (m, 6H), 3.70 (s, 3H), 3.85 (m, 2H), 6.8 (d, J=8.5 Hz, 1H), 6.9 (m, 2H), 7.25 (dd, J=8.5 Hz, 2.1 Hz, 1H), 7.55 (d, J=2.1 Hz, 1H), 7.8 (m, 1H); ¹³C NMR (75.46 MHz, CDCl3): δ 41.13, 45.34, 45.78, 46.32, 55.68, 105.77 (J=25.7 Hz), 112.05 (J=3.8 Hz), 112.61, 113.21, 121.45 (J=3.8 Hz), 126.49, 130.77, 132.89 (J=10.6 Hz), 133.43, 154.14, 158.1 (J=258.8 Hz), 166.01, 167.1 (J=258.8 Hz); HRMS (ESI) calc for [M+H⁺] C₁₈H₁₈N₂O₄SBrF₂ 475.0139, obsd 475.0134; HPLC purity 97.5%, R_{T} 14.24 min.

### Example 28

### 1N-[(2-methoxy-3-bromobenzoyl)]-4-N-[(2,4-difluorophenyl)sulfonyl]piperazine

The general synthetic method described above affords as white solid from 4N-(2,4-difluorophenylsulfonyl)piperazine (0.26 g, 0.99 mmol), and 2-methoxy-3-bromo benzoyl chloride (0.25 g, 0.99 mmol) and N,N-diisopropyl ethyl amine (0.26 mL, 1.49 mmol) in 10 mL of CH₂Cl₂. The crude product was purified by column chromatography (Cyclohexane/Ethyl acetate 50:50) to give compound (0.36 g, 76%). R_{f}=0.51 (Cyclohexane/Ethyl acetate 50:50).

mp: 152.3° C.; ¹H NMR (300.13 MHz, CDCl3): δ 3.05-3.4 (m, 6H), 3.70 (3, 4H), 3.95 (m, 1H), 6.9 (m, 3H), 7.1 (dd, J=7.6 Hz, 1.6 Hz, 1H), 7.55 (dd, J=7.9 Hz, J=1.6 Hz, 1H), 7.8 (m, 1H); ¹³C NMR (75.46 MHz, CDCl3): δ 41.48, 45.53, 45.94, 46.76, 62.10, 105.9 (J=25.7 Hz), 112.2 (J=3.8 Hz), 117.63, 121.74 (J=3.8 Hz), 125.98, 127.27, 131.44, 132.47 (J=12.8 Hz), 134.87, 152.99, 159.75 (J=258.8 Hz), 165.95 (J=258.8 Hz), 166.78; HRMS (ESI) calc for [M+H⁺] C₁₈H₁₈N₂O₄SBrF₂ 475.0139, obsd 475.0137; HPLC purity 98.3%, R_{T} 14.29 min.

### Example 29

### 1N-[(4-methoxybenzoyl)]-4-N-[(2,4-difluorophenyl)sulfonyl]piperazine

The general synthetic method described above affords as white solid from 4N-(2,4-difluorophenylsulfonyl)piperazine (0.26 g, 0.99 mmol), and 4-methoxybenzoyl chloride (0.17 g, 0.99 mmol) and N,N-diisopropyl ethyl amine (0.26 mL, 1.49 mmol) in 10 mL of CH₂Cl₂. The crude product was purified by column chromatography (Cyclohexane/Ethyl acetate 50:50) to give compound (0.2 g, 51%). R_{f}=0.35 (Cyclohexane/Ethyl acetate 50:50).

mp: 143.3° C.; ¹H NMR (300.13 MHz, CDCl3): δ 3.15 (m, 4H), 3.65 (m, 4H), 3.75 (s, 3H), 6.81-6.86 (m, 2H), 6.88-6.99 (m, 2H), 7.26-7.29 (m, 2H), 7.76-7.83 (m, 1H); ¹³C NMR (75.46 MHz, CDCl3): δ 45.79, 55.38, 106.05 (J=25.7 Hz), 112.2 (J=3.8 Hz), 113.88 (2C), 121.44 (J=3.8 Hz), 126.72, 129.30 (2C), 133.02 (J=2.3 Hz), 158.06 (J=258.8 Hz), 161.19, 165.96 (J=258.8 Hz), 166.78; HRMS (ESI) calc for [M+H⁺] C₁₈H₁₉N₂O₄SF₂ 397.1034, obsd 397.1042; HPLC purity 97.3%, R_{T} 12.98 min.

### Example 30

### 1N-[(4-bromobenzoyl)]-4-N-[(2,4-difluorophenyl)sulfonyl]piperazine

The general synthetic method described above affords as white solid from 4N-(2,4-difluorophenylsulfonyl)piperazine (0.26 g, 0.99 mmol), and 4-bromo benzoyl chloride (0.25 g, 0.99 mmol) and N,N-diisopropyl ethyl amine (0.26 mL, 1.49 mmol) in 10 mL of CH₂Cl₂. The crude product was purified by column chromatography (Cyclohexane/Ethyl acetate 50:50) to give compound (0.24 g, 54%). R_{f}=0.66 (Cyclohexane/Ethyl acetate 50:50).

mp: 164.5° C.; ¹H NMR (300.13 MHz, CDCl3): δ 3.15 (m, 4H), 3.65 (br, 4H), 6.89-7 (m, 2H), 7.17 (m, 2H), 7.48 (m, 2H), 7.76-7.86 (m, 1H); HRMS (ESI) calc for [M+H⁺] C₁₇H₁₆N₂O₃SBrF₂ 445.0033, obsd 445.0057; HPLC purity 96.56%, R_{T} 14.29 min.

### Example 31

### 1N-[(3-methoxybenzoyl)]-4-N-[(2,4-difluorophenyl)sulfonyl]piperazine

The general synthetic method described above affords as white solid from 4N-(2,4-difluorophenylsulfonyl)piperazine (0.26 g, 0.99 mmol), and 3-methoxybenzoyl chloride (0.17 g, 0.99 mmol) and N,N-diisopropyl ethyl amine (0.26 mL, 1.49 mmol) in 10 mL of CH₂Cl₂. The crude product was purified by column chromatography (Cyclohexane/Ethyl acetate 50:50) to give compound (0.22 g, 56%). R_{f}=0.42 (Cyclohexane/Ethyl acetate 50:50).

¹H NMR (300.13 MHz, CDCl3): δ 3.3 (br, 4H), 3.7-4.9 (m, 7H), 7 (m, 2H), 7.05-7.17 (m, 3H), 7.41 (m, 1H), 7.96 (m, 1H). ¹³C NMR (75.46 MHz, CDCl3): δ 45.78 (4C), 55.39, 106 (J=25.7 Hz), 112.3 (J=3.8 Hz), 112.69, 115.84, 119.03, 121.53 (J=3.8 Hz), 129.78, 133.02 (J=2.3 Hz), 136.07, 158.2 (J=258.8 Hz), 159.75, 165.82 (J=258.8 Hz), 170.3; HRMS (ESI) calc for [M+H¹] C₁₈H₁₉N₂O₄SF₂ 397.1034, obs 397.1024.

### Example 32

### 1N-[(2-methoxybenzoyl)]-4-N-[(2,4-difluorophenyl)sulfonyl]piperazine

The general synthetic method described above affords as white solid from 4N-(2,4-difluorophenylsulfonyl)piperazine (0.26 g, 0.99 mmol), and 2-methoxybenzoyl chloride (0.17 g, 0.99 mmol) and N,N-diisopropyl ethyl amine (0.26 mL, 1.49 mmol) in 10 mL of CH₂Cl₂. The crude product was purified by column chromatography (Cyclohexane/Ethyl acetate 30:70) to give compound (0.2 g, 51%). R_{f}=0.57 (Cyclohexane/Ethyl acetate 30:70).

mp: 140.8° C.; ¹H NMR (300.13 MHz, CDCl3): δ 3.05-3.5 (m, 6H), 3.75 (s, 3H), 3.9 (m, 2H), 6.9 (d, J=8.3 Hz, 1H), 6.93-7.05 (m, 3H), 7.2 (dd, J=7.5 Hz and 1.7 Hz, 1H), 7.3 (m, 1H), 7.85 (m, 1H). ¹³C NMR (75.46 MHz, CDCl3): δ 41.17, 45.57, 45.99, 46.47, 55.45, 105.87 (J=25.7 Hz), 110.94, 112.16 (J=3.8 Hz), 121.13, 121.57 (J=3.8 Hz), 124.76, 128.13, 130.92, 133.05 (J=2.3 Hz), 155.15, 159.75 (J=258.8 Hz), 165.9 (J=258.8 Hz), 167.89; HRMS (ESI) calc for [M+H⁺] C₁₈H₁₉N₂O₄SF₂ 397.1034, obsd 397.1022; HPLC purity 96.99%, R_{T} 12.83 min.

### Example 33

### 1N-[isonicotinoyl]-4-N-[(2,4-difluorophenyl)sulfonyl]piperazine

The general synthetic method described above affords as white solid from 4N-(2,4-difluorophenylsulfonyl)piperazine (0.26 g, 0.98 mmol), and isonicotinoyl chloride (0.14 g, 0.98 mmol) and N,N-diisopropyl ethyl amine (0.25 mL, 1.49 mmol) in 10 mL of CH₂Cl₂. The crude product was purified by column chromatography (Cyclohexane/Acetone 30:70) to give compound (0.2 g, 56%). R_{f}=0.51 (Cyclohexane/Acetone 30:70).

mp: 130.1° C.; ¹H NMR (300.13 MHz, CDCl3): δ 3.2 (m, 4H), 3.6 (m, 4H), 6.9 (m, 2H), 7.3 (m, 1H), 7.6 (m, 1H), 7.8 (m, 1H), 8.5 (m, 2H); HRMS (ESI) calc for [M+H⁺] C₁₆H₁₆N₃O₃SF₂ 368.0880, obsd 368.0886; HPLC purity 81.02%, R_{T} 9.54 min.

### Example 34

### 1N-[nicotinoyl]-4-N-[(2,4-difluorophenyl)sulfonyl]piperazine

The general synthetic method described above affords as white solid from 4N-(2,4-difluorophenylsulfonyl)piperazine (0.26 g, 0.98 mmol), and nicotinoyl chloride (0.14 g, 0.98 mmol) and N,N-diisopropyl ethyl amine (0.25 mL, 1.49 mmol) in 10 mL of CH₂Cl₂. The crude product was purified by column chromatography (Cyclohexane/Acetone 30:70) to give compound (0.19 g, 53%). R_{f}=0.40 (Cyclohexane/Acetone 30:70).

mp: 148° C.; ¹H NMR (300.13 MHz, CDCl3): δ 3.10 (m, 4H), 3.4 (m, 2H), 3.8 (m, 2H), 6.8 (m, 2H), 7.1 (m, 2H), 7.8 (m, 1H), 8.6 (dd, J=4.35 Hz and J=1.6 Hz); HRMS (ESI) calc for [M+H⁺] C₁₆H₁₆N₃O₃SF₂ 368.0880, obsd 368.0881; HPLC purity 86.6%, R_{T} 9.37 min.

### Example 35

### 1N-[picolinoyl]-4-N-[(2,4-difluorophenyl)sulfonyl]piperazine

The general synthetic method described above affords as white solid from 4N-(2,4-difluorophenylsulfonyl)piperazine (0.26 g, 0.98 mmol), and picolinoyl chloride (0.14 g, 0.98 mmol) and N,N-diisopropyl ethyl amine (0.25 mL, 1.49 mmol) in 10 mL of CH₂Cl₂. The crude product was purified by column chromatography (Cyclohexane/Acetone 30:70) to give compound (0.2 g, 56%). R_{f}=0.76 (Cyclohexane/Acetone 30:70).

mp: 173.6° C. ¹H NMR (300.13 MHz, CDCl3): δ 3.2 (m, 4H), 3.7 (m, 2H), 3.8 (m, 2H), 6.9 (m, 2H), 7.25 (m, 1H), 7.6 (dd, J=, 1H), 7.8 (m, 2H), 8.5 (m, 1H); ¹³C NMR (75.46 MHz, CDCl3): δ 42.07, 45.56, 46.14, 46.86, 105.99 (J=25.7 Hz), 112.23 (J=3.8 Hz), 122.2 (J=3.8 Hz), 124.48, 124.99, 133.04 (J=2.3 Hz), 137.30, 148.17, 153.13, 159.77 (J=258.8 Hz), 165.43 (J=258.8 Hz), 167.42; HRMS (ESI) calc for [M+H⁺] C₁₆H₁₆N₃O₃SF₂ 368.0879, obsd 368.0881; HPLC purity 94.23% R_{T} 13.12 min.

### Example 36

### 1N-[3-thiophenecarbonyl]-4-N-[(2,4-difluorophenyl)sulfonyl]piperazine

The general synthetic method described above affords as white solid from 4N-(2,4-difluorophenylsulfonyl)piperazine (0.25 g, 0.94 mmol), and 3-thiophenecarboxylic acyl chloride (0.14 g, 0.94 mmol) and N,N-diisopropyl ethyl amine (0.25 mL, 1.41 mmol) in 10 mL of CH₂Cl₂. The crude product was purified by column chromatography (Cyclohexane/Ethyl acetate 50:50) to give compound (0.175 mg, 50%). R_{f}=0.43 (Cyclohexane/Ethyl acetate 50:50).

mp: 157.3° C.; ¹H NMR (300.13 MHz, CDCl3): δ 3.2 (m, 4H), 3.8 (m, 4H), 7 (m, 2H), 7.15 (dd, J=1.26 and 5 Hz, 1H), 7.35 (dd, J=2.95 and 5 Hz, 1H), 7.5 (dd, J=1.26 and 2.95 Hz, 1H), 7.95 (m, 1H). ¹³C NMR (75.46 MHz, CDCl3): δ 45.58 (4C), 105.76 (J=25.7 Hz), 111.99 (J=3.8 Hz), 121.2 (J=3.8 Hz), 126.22, 126.62, 126.97, 132.81 (J=2.3 Hz), 135.22, 157.8 (J=258.8 Hz), 165.52 (J=258.8 Hz), 165.62; HRMS (ESI) calc for [M+H⁺] C₁₅H₁₅N₂O₃S₂F₂ 373.0492, obsd 373.0484; HPLC purity 100%, R_{T} 12.26 min.

### Example 37

### 1N-[4-thiazolecarbonyl]-4-N-[(2,4-difluorophenyl)sulfonyl]piperazine

The general synthetic method described above affords as white solid from 4N-(2,4-difluorophenylsulfonyl)piperazine (0.24 g, 0.93 mmol), and 4-thiazole carboxylic acyl chloride (0.14 g, 0.93 mmol) and N,N-diisopropyl ethyl amine (0.24 mL, 1.39 mmol) in 10 mL of CH₂Cl₂. The crude product was purified by column chromatography (Cyclohexane/Ethyl acetate 50:50) to give compound (0.22 g, 63%). R_{f}=0.17 (Cyclohexane/Ethyl acetate 50:50).

mp: 196° C.; ¹H NMR (300.13 MHz, CDCl3): δ 3.2 (m, 4H), 4 (m, 4H), 6.8 (m, 2H), 7.8 (m, 1H), 8 (d, J=2.2 Hz, 1H), 8.7 (d, J=2.2 Hz, 1H). ¹³C NMR (75.46 MHz, CDCl3): δ42.21, 45.62, 46.26, 46.73, 105.96 (J=25.7 Hz), 112.17 (J=3.8 Hz), 121.35 (J=3.8 Hz), 125.89, 133.05 (J=2.3 Hz), 150.92, 152.06, 159.81 (J=258.8 Hz), 162.31, 165.93 (J=258.8 Hz); HRMS (ESI) calc for [M+H⁺] C₁₄H₁₄N₃O₃S₂F₂ 374.0445, obsd 374.0453; HPLC purity 99.19%, R_{T} 10.82 min.

TABLE I
(IIa)

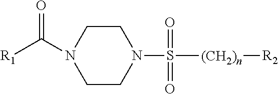

| | | | | mP |
|---|---|---|---|---|
| example | R₁ | R₂ | n | ° C. |
| 1 | 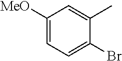 | 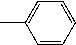 | 0 | 180 |
| 2 | 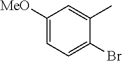 | 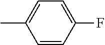 | 0 | 202 |
| 3 | 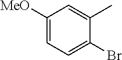 | 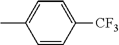 | 0 | 155 |
| 4 | 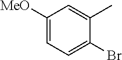 | 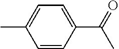 | 0 | 143 |
| 5 | 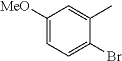 | 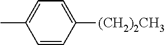 | 0 | 134 |
| 6 | 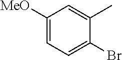 | 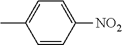 | 0 | 151 |
| 7 | 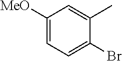 | 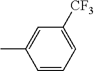 | 0 | 154 |
| 8 | 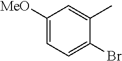 | 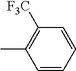 | 0 | 136.5 |
| 9 | 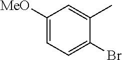 | 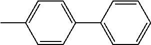 | 0 | 100 |
| 10 | 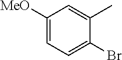 | 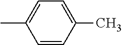 | 0 | 178 |
| 11 | 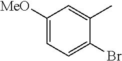 | 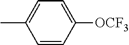 | 0 | 110.4 |
| 12 | 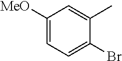 | 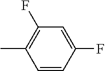 | 0 | 194 |
| 13 | 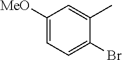 | 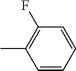 | 0 | 167.3 |
| 14 | 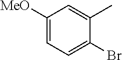 | 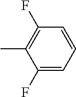 | 0 | 154.5 |
| 15 | 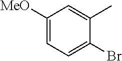 | 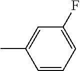 | 0 | 145 |
| 16 | 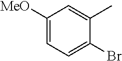 | 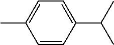 | 0 | 123 |
| 17 | 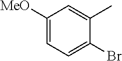 | 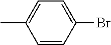 | 0 | 180 |
| 18 | 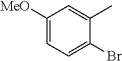 | 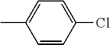 | 0 | 188 |
| 19 | 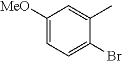 | 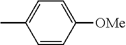 | 0 | 187 |
| 20 | 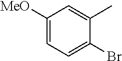 | 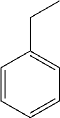 | 1 | 112 |
| 21 | 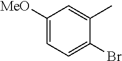 | 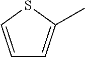 | 0 | 155.8 |
| 22 | 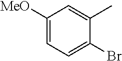 | 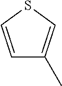 | 0 | 156.9 |
| 23 | 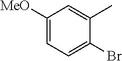 | 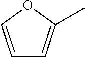 | 0 | 132.8 |
| 24 | 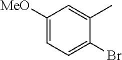 | 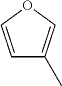 | 0 | 162.3 |
| 25 | 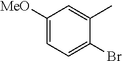 | 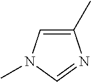 | 0 | 190.2 |
| 26 | 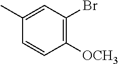 | 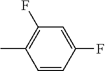 | 0 | 166.7 |
| 27 | 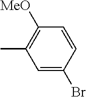 | 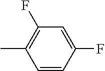 | 0 | 171.2 |
| 28 | 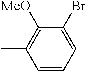 | 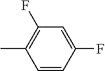 | 0 | 152.3 |
| 29 | 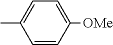 | 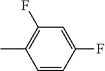 | 0 | 143.3 |
| 30 | 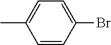 | 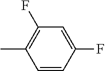 | 0 | 164.5 |
| 31 | 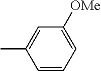 | 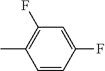 | 0 | — |
| 32 | 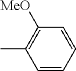 | 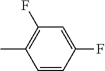 | 0 | 140.8 |
| 33 | 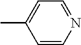 | 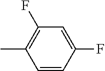 | 0 | 130.1 |
| 34 | 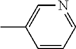 | 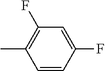 | 0 | 148 |
| 35 | 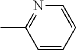 | 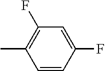 | 0 | 173.6 |
| 36 | 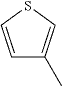 | 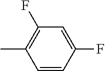 | 0 | 157.3 |
| 37 | 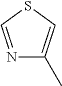 | 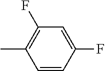 | 0 | 196 |

### Example 38

### Biological Experiments

The compound 1N-[(2-bromo-5-methoxybenzoyl)]-4N-[(phenylpropan)pyridine-3-carboxamide)]piperazine (BZ36), and, depending on the biological assays, compounds of the invention described in previous examples 1 to 37 have been subjected to biological assay(s) which demonstrate their relevance as active substances in therapy and in particular in the treatment of prostate cancer.

Material and Methods

Compound 1N-[(2-bromo-5-methoxybenzoyl)]-4N-[(phenylpropan)pyridine-3-carboxamide)]piperazine (BZ36) was prepared according to the method described in the patent US 2005/0119251 A1.

Human Prostate Tissue Samples

Human prostate tissue was collected from consenting patients, after protocol approval by the local ethics committee (CHU Henry Mondor, Creteil) and characterisation by urological pathologist. Localized prostate cancer specimens from the peripheral zone of the prostate were obtained from men who had radical prostatectomy as treatment for their prostate cancer, Gleason≧7 (n=10). Non tumoral prostate specimens were obtained from men with begnin hyperplasia of the prostate (HBP) who had radical prostatectomy (n=10).

Cell Culture, Transient Transfections and RNA Interference Experiments

The benign PNT2 prostate cell line, the androgen-sensitive LNCaP and the androgen-independent C4-2 human prostate carcinoma cell lines were purchased from American Type Culture Collection (Manassas, Va.).

Monolayer cell cultures were maintained in a RPMI 1640 media M 1-glutamine (Invitrogen, Cergy, France) supplemented with 10% fetal calf serum (FCS), 100 U/ml penicillin, 100 μg/ml streptomycin, 10 mM HEPES and 1.0 mM sodium pyruvate (Invitrogen) at 37° C. in 5% CO₂.

Primary MEFs were obtained from embryos at embryonic day 13.5 by standard methods.

Monolayer cell cultures were grown in Dulbecco's Modified Eagle's Medium (DMEM) supplemented with 25 mM glucose and 10% FCS.

The transcriptional activity of the β-catenin-Tcf4 complex was analysed by performing transient transfections with 0.25 μg TCF/LEF-1 reporter (pTOP-FLASH) or control vector (pFOP-FLASH).

Luciferase activities in cell lysates were normalized relative to the β-galactosidase activity to correct for differences in transfection efficiency.

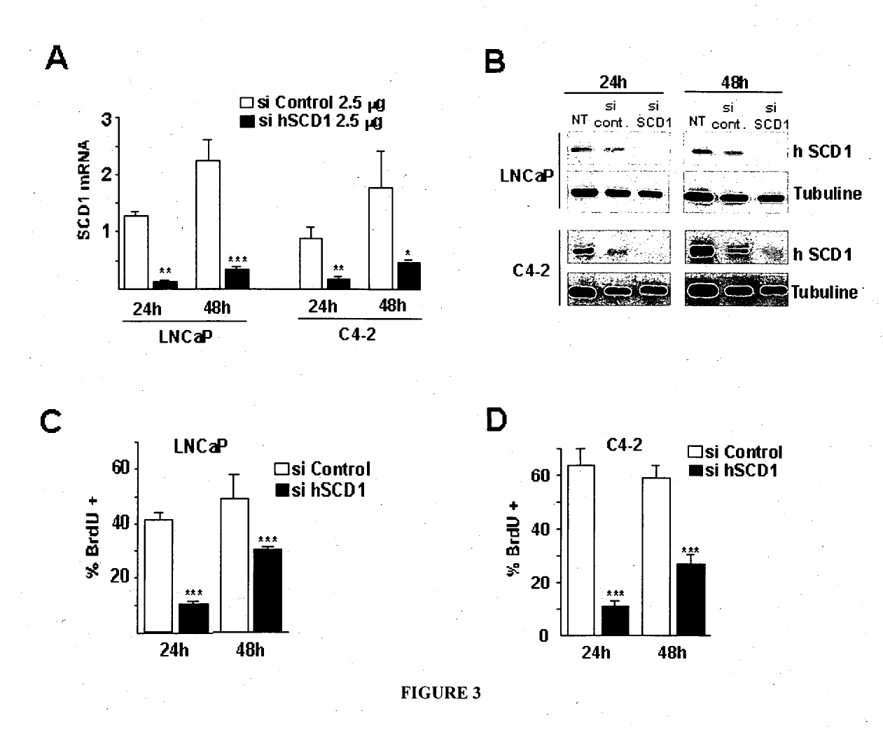

FIG. 3 shows representative results of at least two independent experiments performed in triplicate.

For small interfering (si) RNA experiments, transfection of LNCaP or C4-2 cells was carried out with pre-designed ON-TARGET plus siRNA oligonucleotides control or targeting the human SCD-1 sequence GCACAUCAACUUCACCACA (Dharmacon, Lafayette, Co, USA).

Nucleofaction of cells with 2.5 μg siRNA were performed on 2×10⁶ cells using Amaxa nucleofactor R kit (Lonza, Cologne, Germany). Twenty-four hours and 48 hours after transfection, cells were processed for cell proliferation by BrdU staining and harvested for RNA and protein analysis.

Animal Experiments

Male athymic nude Mice (Foxn1 nu/nu) (Harlan, Grannat, France) were used at the age of 7 weeks (weight 25-30 g). All procedures were performed in compliance with the European Convention for the Protection of Vertebrate Animals Used for Experimentation (animal house agreement # B-34-172-27, authorization for animal experimentation #34.324). Experiments were done at least twice for each tested condition. Animals were sacrificed before they became compromised. Xenografts were established by subcutaneously injecting 2×10⁶ LNCaP cells, 2×10⁶ C4-2 cells or 5×10⁵ MEFs SV40 in 100 μl of a Matrigel solution. For curative experiments, tumors were allowed to growth until they were measurable with a caliper. In each group, the mice were randomized and given SCD-1 inhibitor at 80 mg/kg in 100 μl of a labrafil-DMA-tween⁸⁰ solution (89:10:1) (treated group) or vehicle alone (control group), by daily intra-peritoneal (i.p.) injection 5 days of week. For preventive experiments, the mice were treated daily for 7 days before the day of xenograft. Tumor volume measurements were taken twice to three times a week and calculated according to the formula: length×width×height×0.5236. Data are expressed as the mean tumor volume or as fold of the start point tumor volume. For survival analysis, animals bearing pre-established C4-2 tumors were treated with BZ36 at 80 mg/kg or 160 mg/kg or with vehicle by daily i.p. injection 5 days of week. All mice were monitored for survival until tumor volume had reached 2000 mm³ or until death. Analysis of survival was conducted by a log-rank test based on the Kaplan-Meier method. At euthanasia, tumors were excised and fixed in 4% formalin for immunohistological analyses.

Rna Isolation, Reverse Transcription and Quantitative Real-Time PCR:

RNA was extracted with the use of TRI-Reagent (Euromedex, Mundolsheim, France) according to the manufacturers' recommendations. Reverse transcription of total RNA was performed at 37° C. using the M-MLV reverse transcriptase (Invitrogen) and random hexanucleotide primers (Promega, Madison, Wis.), followed by a 15 min inactivation at 70° C. Quantitative PCR was conducted using the primers specific for human SCD1 and SYBR Green Light Cycler Master Mix (Eurofins MWG Operon, Roissy CDG). Measurement and analysis of gene expression were performed using the ABI Prism 7300 Sequence detection System software (Applied Biosystems), under the following conditions: 2 minutes at 50° C. and 10 minutes at 95° C.; and then 40 cycles of 15 seconds at 95° C. and 1 minute at 60° C. The relative content of cDNA samples was normalized by substracting the threshold cycle (Cₜ) of the endogenous 18S reference gene to the target gene (ΔCₜ═Cₜ of target gene −Cₜ of 18S). Values are expressed as the relative mRNA level of specific gene expression as obtained using the formula 2^{-(ΔCt)}.

C4-2 cells treated with the compound tested at 25 μM were analysed for the expression profiles of 84 genes related to Wnt-mediated signal transduction by using Human WNT Signaling Pathway PCR Array according to the manufacturers' recommendations (Tebu-Bio, Le Perray en Yvelines, France). The relative content of cDNA samples was normalized with the endogenous β2M, HPRT1, RPL13 and GAPDH reference genes, and the relative mRNA level was calculated according to the formula 2^{-(ΔCt)}. Values are expressed as the fold change in relative mRNA level following treatment of cells with the tested compound at 25 μM, as compared to control.

Proliferation Assay

LNCaP, C4-2, PNT2 or MEFs cells were seeded in triplicate 24-wells dishes at a density of 25×10⁴ cells/dish. At 24 h, 48 h, 72 h and 96 h following addition of increasing concentrations of the tested compound in DMSO or DMSO alone, cells were trypsinized, pelleted by centrifugation at 1200 rpm for 5 min, resuspended in 500 μl of culture medium and counted in an hemocytometer.

MTT Assay

LNCaP, C4-2 or PNT2 cells were seeded in triplicate 24-wells dishes at a density of 25×10⁴ cells/dish and cells viability was tested after treatment with increasing concentrations of the tested compound for 48 h. After 48 h, medium was removed and 250 μl of a 5 mg/ml MTT (Sigma, St Louis, Mo., USA) solution in PBS was added to each well. After 4 h incubation at 37° C., the MTT solution was removed, 200 μl of DMSO (Sigma) was added and cells were incubated for 5 min. Two hundred microliters of each samples was distributed in 96-well plates for an optical density reading at 540 nm.

Flow Cytometry Analysis of Cell Cycle and Apoptosis

For cell cycle analysis, LNCaP, C4-2 or PNT2 cells were plated at a density of 25×10⁵ cells/dish in 6-wells dishes and treated with increasing concentrations of the tested compound for 24 h or 48 h. Cells were then rinsed in PBS, pelleted at 400 g for 5 min and maintained on ice for 20 min before resuspension in a 25 propidium iodide (Sigma) solution. Cells were kept overnight at 4° C. and the percentages of cells in G1, S and G2-M phases of the cell cycle were measured with a Coulter Epics XL™ flow cytometer (Becton Dickinson) using 488-nm laser excitation. For apoptosis experiment, MEFs were treated with the tested compound at 25 μM for 24 h, then rinced in PBS, pelleted at 400 g for 5 min and stained with annexinV-FITC (Roche Diagnostics, Meylan, France) for 15 min at 4° C. The percentage of annexin positive cells was immediately analysed using 488-nm laser excitation.

Protein Extracts and Immunoblot Analysis

Protein extracts and sodium dodecyl sulfate (SDS)-polyacrylamide gel electrophoresis (PAGE), electrotransfer and immunoblotting were performed as previously described (34). The primary antibodies rabbit anti-AMPK, rabbit anti-phospho AMPK (Thr172), rabbit anti-Akt and rabbit anti-phospho Akt were purchased from Cell signalling Technology (Ozyme, Saint Quentin Yvelines, France). The primary antibody mouse anti-tubulineα was purchased from Lab Vision (Thermo Fisher Scientific, Microm France, Francheville, France). The primary antibodies rabbit anti-p44/p42 MAPK, rabbit anti-phospho p44/p42 MAPK (Thr202/Tyr204), mouse anti-GSK3α/β, and rabbit anti-phospho GSK3α/β were kindly provided by Dr Gilles Freiss. LNCaP and C4-2 cells treated with the tested compound at 25 μM were analyzed for the relative phosphorylation of 46 kinase phosphorylation sites using human phospho-kinase array kit (Proteome Profiler™) according to the manufacturers' recommendations (R&D systems Europe, Lille, France).

Fatty Acid Analysis

Total prostate tissue lipid were extracted three times with 5 mL choloroform/methanol (2/1) and 500 μL of water. Aliquots of the lipid extracts were dried under gaseous nitrogen and were dissolved in 100 μL of a mixture and were separated by thin layer chromatography in different lipid classes using a hexane/ether/acetic acid (70/30/1; V/V) solvent system. To aid visualization, standarts of triacylglycerol, of cholesteryl esters and phospholipds were co-spotted with samples. Spots were identified under UV light after spraying with 2′,7′-dichlorofluorescein solution in ethanol and comparing with authentic standarts. They were scrapped off the plates and were converted to fatty methyl esters by transesterification with 3 mL of methanol/H₂SO₄ (19/1; V/V) at 90° C. for 30 min. The different solutions were neutralized with an 1 mL of aqueous solution of 10% of K₂CO₃ and fatty methyl esters were extracted with 5 mL of hexane. The fatty methyl esters were dried under gaseous nitrogen and were subjected to gas chromatography (GC) and identified by comparaison with standarts. (Sigma Chemicals, St. Louis, Mo.). GC was conducted with a Thermo GC fitted with a flame ionization detector. A supelcowax-10 fused silica capillary column (60m×0.32 mm i.d, 0.25 μM film thickness) was used and oven temperature was programmed from 50° C. to 200° C., increased 20° C. per minute, held for 50 min, increased 10° C. per min to 220° C., and held for 30 min.

Determination of SCD Activity

Subconfluent LNCaP, C4-2, PNT2 of Ras SV40 MEFs cells grown in 6-wells plate were incubated with the tested inhibitor at 25 μM for 2 h in serum and fatty acid-free DMEM media supplemented 0.2% BSA. In this environment, the cells are solely dependent on endogenous fatty acid synthesis for production of storage, structural and signaling lipids. Trace amount of [¹⁴C] palmitic acid were then added to the culture (0.5 μCi/well), and cells were incubated for 6 more hours. At the end of the incubation, total cell lipids were extracted and saponified, then released fatty acids were esterified with boron trifluoride in methanol for 90 min at 100° C. The derived methyl esters were separated by argentation TLC (Thermo Fisher Scientific) following the procedure of Wilson and Sargen, using a solvent phase consisting of hexane:ethyl ether (90:10, by vol) (35). Pure stearic and oleic methyl ester acids were run in parallel to the samples. Air-dried plates were scanned on a Phosphorlmager and fatty acid spots on TLC were analysed with Phosphorlmager software. SCD activity was expressed as the ratio of palmitoleic on palmitic methyl ester acids and normalized to cellular DNA content.

Measurement of De Novo Fatty Acid Synthesis

The tested inhibitor was added overnight at a final concentration of 25 μM to subconfluent cultures of cells grown in 6-wells plate in serum and fatty acid-free DMEM media supplemented with 0.2% BSA. Cultures were then labeled in triplicate with 1.0 μCi of [U—¹⁴C]-palmitate or -stearate for 6 h, and total lipids were folch extracted with chloroform/methanol. Labeled lipids were subjected to TLC in hexane/diethyl ether/acetic acid 90:10:1 (V/V), to separate cholesterol ester, triglycerides and phospholipids. Standards were run for each of the lipid classes. After chromatography, labeled lipid classes were quantified by scintillation counting and radioactivity was normalized to DNA content.

PI(3,4,5)P₃ Measurement

The production of PI(3,4,5)P₃ (PIP3) in LNCaP and C4-2 prostate cancer cells was measured 24 h following exposure to control medium or to medium supplemented with the inhibitor tested at 25 μM. The levels of produced PIP3 were quantified after cellular lipids extraction using a PI(3,4,5)P₃ mass ELISA kit according to the manufacturer's instruction.

Immunofluorescence (IF) and Immunohistochemistry (1HC) of hSCD-1

Immunohistochemical analysis of SCD-1 expression was performed using high-density Tissue Microarray (TMA) slide (Accumax array) with 39 prostate adenocarcinoma spots from different patients (Gleason scores from 5 to 9) with corresponding normal tissues. Immunohistochemical analysis of pcna expression was performed on 5 μM paraffin-embedded sections of LNCaP, C4-2 of Ras SV40 MEFs tumor xenografts. Briefly, after antigen retrieval, deparaffinized sections were blocked of Fc receptors with PBS containing 5% goat serum and then incubated with corresponding anti-SCD-1 mouse antibody, 1:50 (Abcam, Paris, France) or anti-pcna mouse antibody, 1:50 (Santa Cruz) in PBS-Tween 0.1%, overnight at 4° C. SCD-1 staining was revealed with a peroxidase-conjugated anti-mouse secondary antibody, 1:100 (Jackson Immunoresearch) and the DAB chromogen (DAKO, Glostrup, Denmark) as substrate. Sections were counterstained with Mayer's hematoxylin. Pcna staining was revealed by immunofluorescence using a FITC-conjugated anti-mouse secondary antibody, 1:150 (Jackson Immunoresearch). Sections were mounted in mowiol and analysed rapidly.

Immunocytochemistry

Immunocytochemical analysis of SCD-1 expression was performed on PNT2, LNCaP, and C4-2 cells grown on coverslip. Briefly, after fixation in 4% PFA and permeabilization with 0.5% Triton X-100, cells were incubated with blocking buffer (PBS-1% BSA). SCD1 staining was detected with an anti-SCD-1 mouse primary antibody, 1:50 (Abcam) for 1 hour at 37° C. and revealed with a FITC-conjugated anti-mouse secondary antibody (Jackson Immunoresearch), 1:150 for 30 min at 37° C. Slides were mounted in mowiol and analysed rapidly.

BrDU Staining

LNCaP and C4-2 cells grown on coverslips were incubated for 4 hours with BrdU (100 μM final) at 24 hours and 48 hours following SCD-1 knock-down. Cells were then fixed and permeabilized with cold methanol for 10 min at −20° C. After 3 washes with PBS, DNA was denaturated with 4NHCL for 10 min at RT, and cells were incubated with blocking buffer (PBS-1% BSA). BrDU was then detected with anti-BrDU monoclonal antibody 1:50 (Dako, Carpinteria, Calif.) for 1 hour at 37° C. After 3 washes with PBS, cells were incubated with an FITC-conjugated anti-mouse secondary antibody 1:150 (Jackson Immunoresearch) for 30 min at 37° C., and slides were mounted in mowiol.

Statistical analysis were performed with unpaired Student's t-test. Differences were considered statistically significant at p<0.05. (* p<0.05; ** p<0.01 and *** p<0.001).

Results

MUFA Content and SCD-1 Expression are Increased During Prostate Cancer Progression

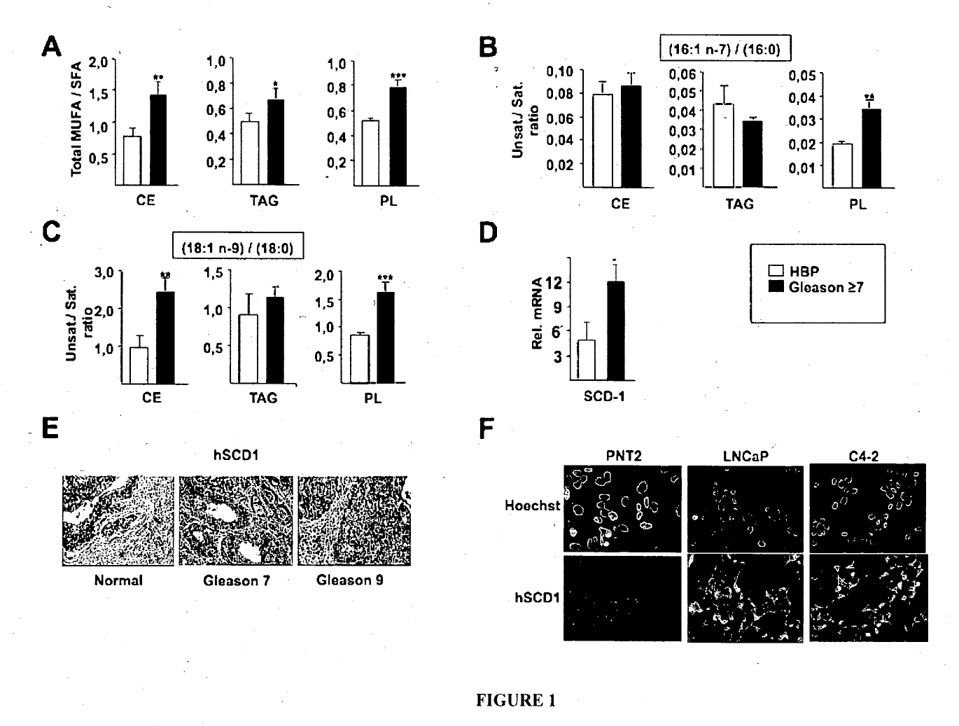

As a first approach to fatty acid FA composition was measured during prostate cancer progression. The ratios of total MUFA to saturated fatty acids (SFA) was significantly increased in cholesterol esters (CE, triacylglycerides (TAG) and phospholipids (PL) lipid fractions in human prostate cancer tissue samples with Gleason score ≧7 (FIG. 1A), suggesting the participation of desaturase enzymes. Interestingly, among the analyzed MUFAs, those produced by a Δ9-desaturase activity, in majority palmitoleate (16:1n-7) and oleate (18:1n-9), were the most abundant in all lipid subclasses (data not shown). Furthermore, the specific desaturation indexes 16:1n-7/16:0 (FIG. 1B) and 18:1n-9/18:0 (FIG. 1C) were increased in human prostate samples, supporting the participation of the Δ9-desaturase enzyme.

Consistent with this observation, stearoyl-CoA Δ9-desaturase 1 (SCD-1) was increased in cancer, compared to normal human prostates both at mRNA (FIG. 1D) and protein levels (FIG. 1E), as analyzed by QPCR and immunohistochemistry (1HC) studies respectively.

SCD-1 expression was also increased, as measured by immunofluorescence in the human prostate cancer cell lines LNCaP and C4-2, compared to the non-tumoral PNT2 benign prostate cell line (FIG. 1F).

Pharmacological and Genetic Inhibition of SCD-1 activity by 1N-[(2-bromo-5-methoxybenzoyl)]-4N-[(phenylpropan)pyridine-3-carboxamide)]piperazine Induces Growth Arrest of LNCaP Cell Lines In Vitro

Compound 1N-[(2-bromo-5-methoxybenzoyl)]-4N-[(phenylpropan)pyridine-3-carboxamide)]piperazine (BZ36) was tested.

The efficacy of the tested compound to inhibit SCD-1 activity has been evaluated by measuring the conversion of exogenous [¹⁴C] saturated palmitic acid (16:0) to monounsaturated palmitoleic acid (16:1n-7). Moreover, the effects of inhibition of SCD-1 activity on lipid synthesis and proliferation in prostate cancer cell lines were analysed.

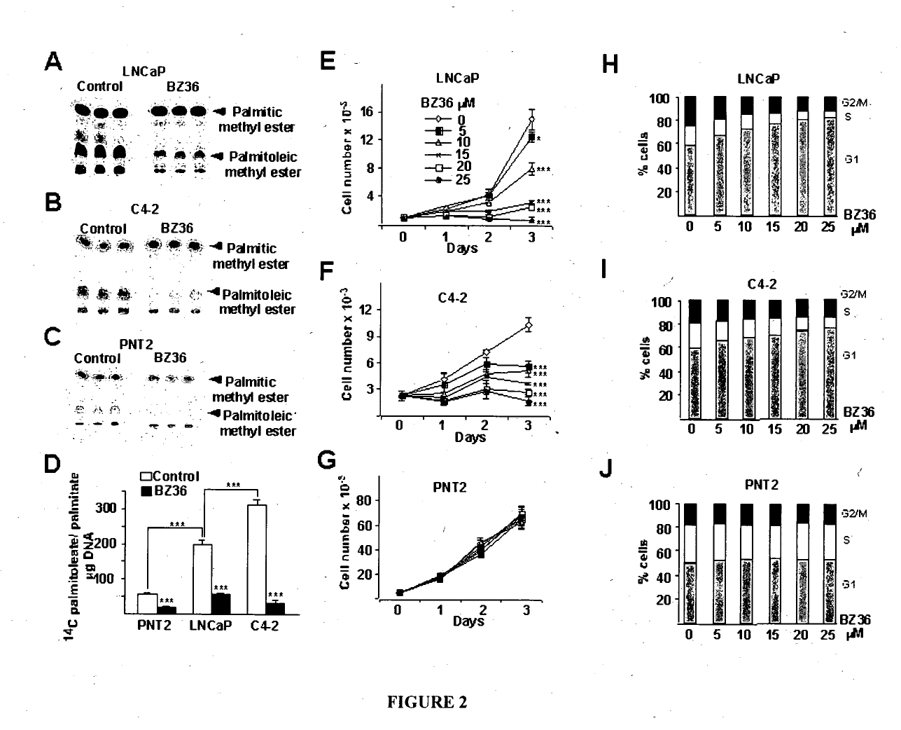

In all three cell lines tested a marked reduction of the labelled monounsaturated palmitoleic acid was observed in the cells with BZ36 treated, compared to control cells (FIG. 2A-D).

Interestingly, basal SCD-1 activity was progressively increased from non-tumoral PNT2 to androgen independent C4-2 cell lines, further suggesting the implication of SCD-1 in prostate cancer progression (FIG. 2D). Inhibition of SCD-1 activity with the tested compound correlated with a dose dependent decrease in cell proliferation of LNCaP, and C4-2 cancer cells, reaching 100% inhibition at the maximal dose used (FIG. 2E-F).

Flow cytometry analysis further demonstrated the inhibitory effects of the tested compoundin CaP cells, showing accumulation of cells in the G0/G1 phase of the cell cycle, concomitant with a decrease in the S phase (FIG. 2G-I).

Strikingly, no effect in proliferation was observed in the non-cancerous PNT2 cell line, even at a maximal dose. Similar to what observed using SCD1 small molecule inhibitors, genetic SCD-1 inhibition using siRNA technology blocked SCD1 mRNA (FIG. 3A) and protein (FIG. 3B) expressions, and resulted in a marked decrease in proliferation in both LNCaP (FIG. 3C), and C4-2 (FIG. 3D) cell types. These results suggested that, first, SCD-1 activity and lipid synthesis is required in prostate cancer cells in order to proliferate. Second, that non-cancer cell do not require de novo lipid synthesis, and therefore normal cells are not sensitive to inhibition of this pathway. And third, that the inhibitory effects of SCD-1 inhibitors are mediated by SCD-1 since the same effects are observed when SCD-1 is depleted from the cells.

Proliferation of LNCaP and C4-2 cells in presence or absence of 25 μM of compounds prepared according to the examples 1 to 28 was measured by BrDu incorporation following 48 h of treatment with the compounds Inhibition of SCD-1 activity with the indicated tested compounds results in a decrease of the proliferation of C4-2 and LNCaP cells.

The results are disclosed in the following table II and table III.

TABLE II
(IIa)

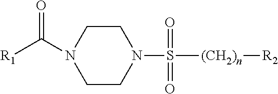

| | | | | % Inhibition of proliferation at |
|---|---|---|---|---|
| example | R₁ | R₂ | n | 25 μM (BrDU) on C4-2 |
| 1 | 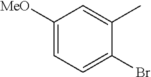 | 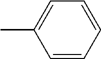 | 0 | 22.7 ± 11.2 |
| 2 | 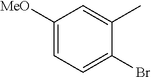 | 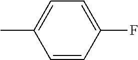 | 0 | 37.2 ± 8.8 |
| 3 | 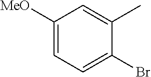 | 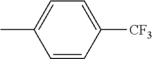 | 0 | 65.8 ± 6.4 |
| 4 | 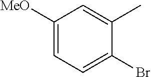 | 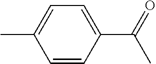 | 0 | 36 ± 5.4 |
| 5 | 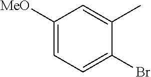 | 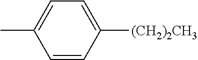 | 0 | 26.8 ± 20.2 |
| 6 | 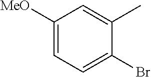 | 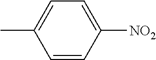 | 0 | 0 |
| 7 | 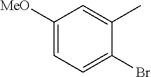 | 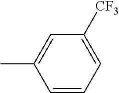 | 0 | 31.6 ± 7.61 |
| 8 | 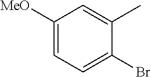 | 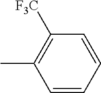 | 0 | 84.4 ± 8.5 |
| 9 | 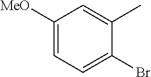 | 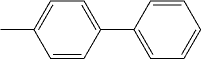 | 0 | 60.5 ± 5.7 |
| 10 | 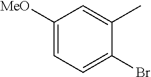 | 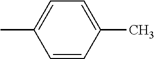 | 0 | 0 |
| 11 | 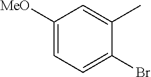 | 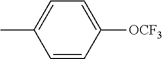 | 0 | 63 ± 4.1 |
| 12 | 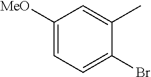 | 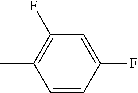 | 0 | 55.2 ± 0.5 |
| 13 | 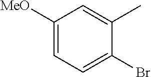 | 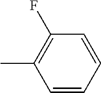 | 0 | 55.6 ± 3.1 |
| 14 | 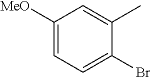 | 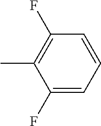 | 0 | 0 |
| 15 | 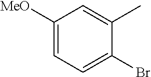 | 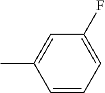 | 0 | 35.7 ± 6 |
| 16 | 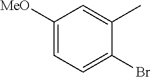 | 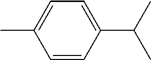 | 0 | 75.4 ± 10.8 |
| 17 | 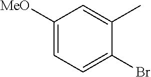 | 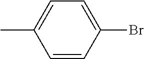 | 0 | 67.6 ± 8.2 |
| 18 | 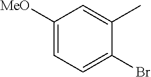 | 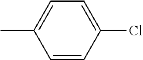 | 0 | 38.1 ± 7.1 |
| 19 | 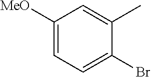 | 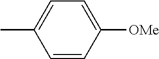 | 0 | 22.4 ± 7.7 |
| 20 | 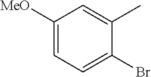 | 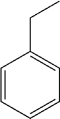 | 1 | 46.3 ± 11.7 |
| 21 | 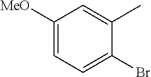 | 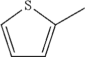 | 0 | 58.3 ± 19.9 |
| 22 | 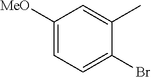 | 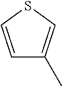 | 0 | 28 ± 2.6 |
| 23 | 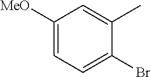 | 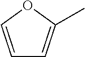 | 0 | 48 ± 14 |
| 24 | 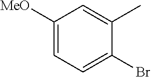 | 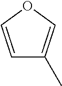 | 0 | 28.4 ± 9.8 |
| 25 | 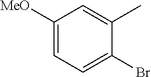 | 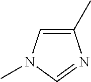 | 0 | 0 |
| 26 | 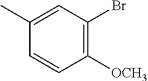 | 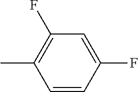 | 0 | 68.2 ± 18.2 |
| 27 | 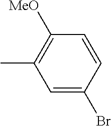 | 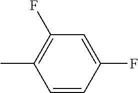 | 0 | 26.2 ± 6.2 |
| 28 | 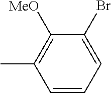 | 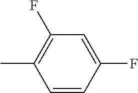 | | 55.4 ± 14 |
| 31 | 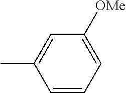 | 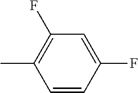 | 0 | 25.3 ± 6.1 |
| 32 | 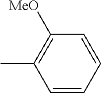 | 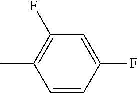 | 0 | 0 |
| 36 | 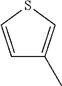 | 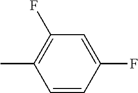 | 0 | 40 ± 7.6 |
| 37 | 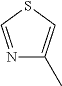 | 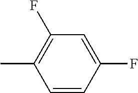 | 0 | 37 ± 7.8 |

TABLE III
(IIa)

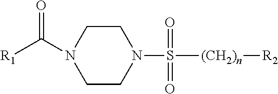

| | | | | % Inhibition of |
|---|---|---|---|---|
| | | | | proliferation at 25 μM |
| Examples | R₁ | R₂ | n | (BrDU) on LNCaP |
| Example 7 | 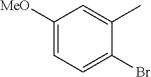 | 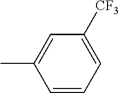 | 0 | 16.7 |
| Example 8 | 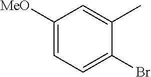 | 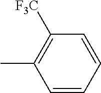 | 0 | 40.1 |
| Example 9 | 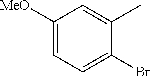 | 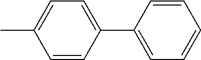 | 0 | 48.3 |
| Example 10 | 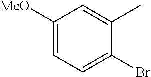 | 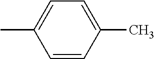 | 0 | 0 |
| Example 11 | 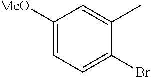 | 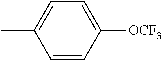 | 0 | 45.5 |
| Example 12 | 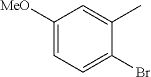 | 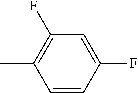 | 0 | 60.6 |
| Example 13 | 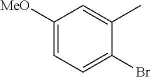 | 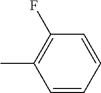 | 0 | 27.1 |
| Example 15 | 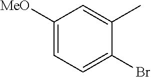 | 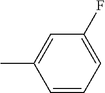 | 0 | 0 |
| Example 18 | 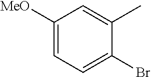 | 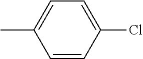 | 0 | 49.7 |
| Example 20 | 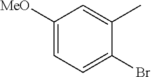 | 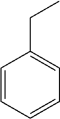 | 1 | 16.8 |

Accordingly, the inhibition of LNCap and C4-2 cells proliferation is also observed with the tested compound.

Percentage of proliferative LNCaP and C4-2 cells in the S phase of the cell cycle following 48 h of treatment in presence or absence of 25 μM of compounds prepared according to the examples 1 to 6 was measured. Inhibition of SCD-1 activity with the indicated tested compounds decreased the percentage of proliferative LNCaP and C₄₋₂ cells in the S phase.

The so-obtained resultst are disclosed in the following table IV.

TABLE IV
(IIa)

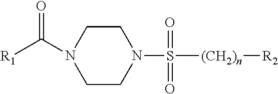

| | | | | % LNCaP cells | % C4-2 cells |
|---|---|---|---|---|---|
| | | | | in phase S | in phase S |
| example | R₁ | R₂ | n | at 25 μM | at 25 μM |
| Cellules | | | | 20 | 22 |
| non | | | | | |
| traitées | | | | | |
| 1 | 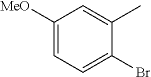 | 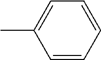 | 0 | 18 | 20.1 |
| 2 | 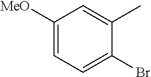 | 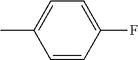 | 0 | 5 | 11.8 |
| 3 | 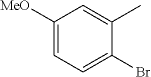 | 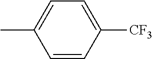 | 0 | 10 | 2.8 |
| 4 | 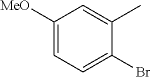 | 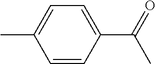 | 0 | 18 | 19.7 |
| 5 | 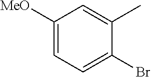 | 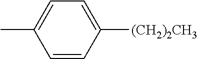 | 0 | 3 | 10.7 |
| 6 | 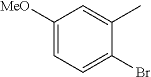 | 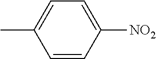 | 0 | 15 | 19.8 |

Percentage of viable C4-2 cells as measured by MTT assay following 48 h of treatment in the presence or absence of 25 μM of compounds prepared according to the examples 1 to 13, 15, 18 and 20 was evaluated. Inhibition of SCD-1 activity with the indicated tested compounds decreased the cell viability.

The results are disclosed in the following table V and are expressed as % of viable treated cells normalized to viable control cells.

TABLE V
(IIa)

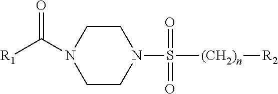

| | | | | Viable C4-2 cells |
|---|---|---|---|---|
| | | | | at 25 μM |
| example | R₁ | R₂ | n | (% of control) |
| Cellules | | | | |
| non | | | | |
| traitées | | | | |
| 1 | 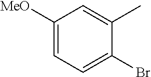 | 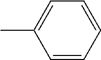 | 0 | 45 |
| 2 | 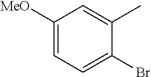 | 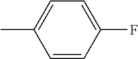 | 0 | 49 |
| 6 | 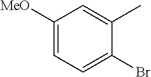 | 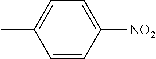 | 0 | 42 |
| 7 | 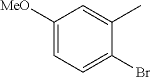 | 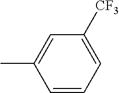 | 0 | 44 |
| 8 | 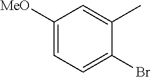 | 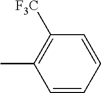 | 0 | 73 |
| 9 | 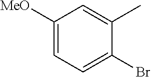 | 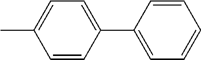 | 0 | 90 |
| 10 | 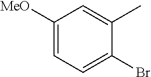 | 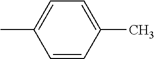 | 0 | 59 |
| 11 | 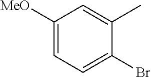 | 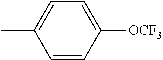 | 0 | 74 |
| 12 | 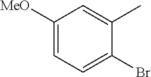 | 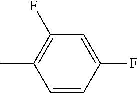 | 0 | 70 |
| 13 | 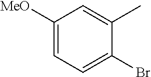 | 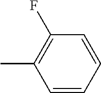 | 0 | 56 |
| 15 | 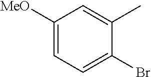 | 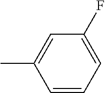 | 0 | 83 |
| 18 | 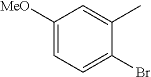 | 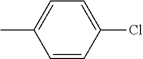 | 0 | 59 |
| 20 | 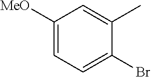 | 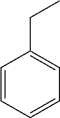 | 1 | 37 |

Inhibition of SCD-1 Activity by 1N-[(2-bromo-5-methoxybenzoyl)]-4N-[(phenylpropan)pyridine-3-carboxamide)]piperazine Decreases De Novo Fatty Acid Synthesis in CaP Cell Lines

The tested compound is the 1N-[(2-bromo-5-methoxybenzoyl)]-4N-[(phenylpropan)pyridine-3-carboxamide)]piperazine (BZ36).

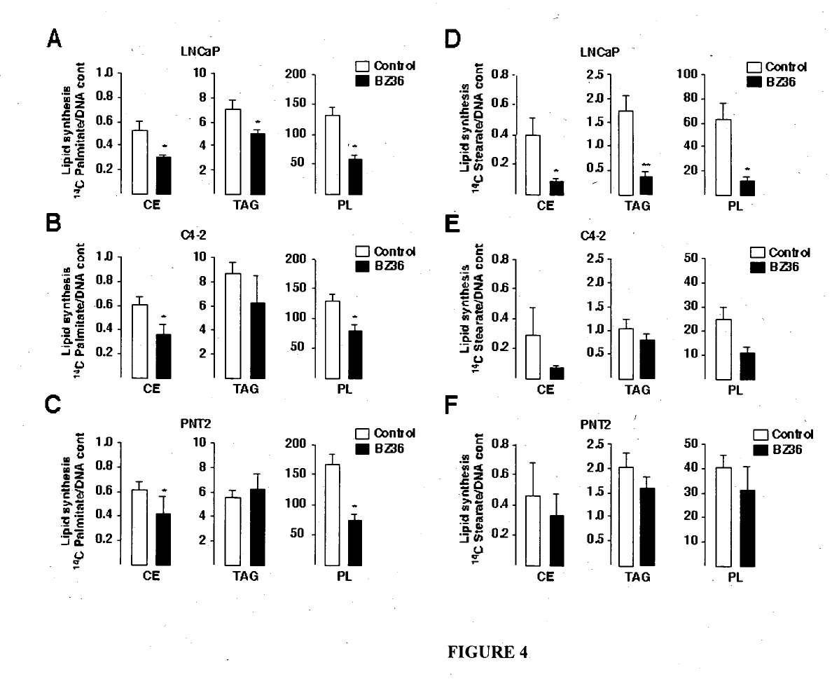

It has also been checked that [U—¹⁴C]-palmitate incorporation into sterol esters, triglycerides and phospholipids, which is a measure of de novo lipid synthesis was respectively inhibited by 39%, 27% and 58% in LNCaP (FIG. 4A), and by 33%, 24% and 41%, respectively in C4-2 cells (FIG. 4B) treated with the tested compound. Similarly, [U—¹⁴C]-stearate incorporation into sterol esters, triglycerides and phospholipids was also inhibited respectively by 77%, 78%, and 81% in LNCaP (FIG. 4D), and by 70%, 19% and 49%, in C4-2 cells (FIG. 4E), treated with the tested compound. Similar results were observed in PNT2 cells (FIG. 4C,F).

SCD1 Inhibition Interferes with Major Signaling Pathways in Prostate Cancer Cells

The tested compound is the 1N-[(2-bromo-5-methoxybenzoyl)]-4N-[(phenylpropan)pyridine-3-carboxamide)]piperazine (BZ36).

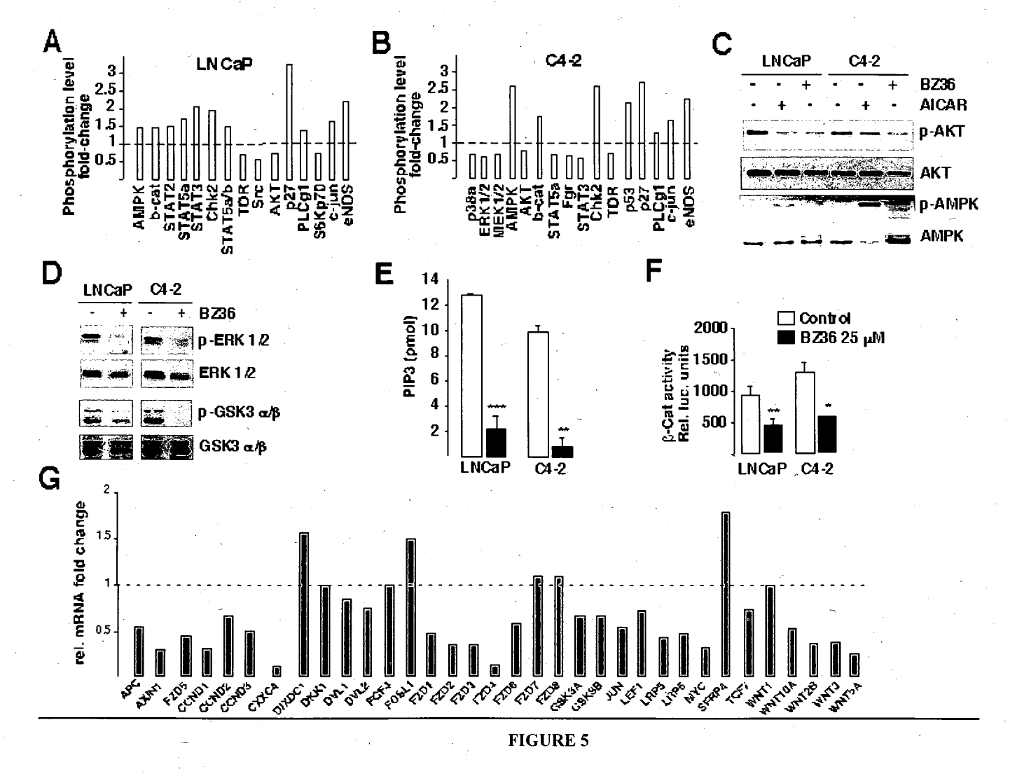

Lipids are important signaling molecules that actively participate in triggering specific phosphorylation pathways. Since inhibition of SCD-1 activity was associated with a significant reduction in de novo lipids synthesis in prostate cancer cells (FIG. 4), we expected also a decrease in these pathways. The relative phosphorylation status of several kinases in both LNCaP and C4-2 cells in response to treatment with the tested compound (BZ36) showed important differences (FIG. 5A-B). Relevant for our study was the decrease in Akt phosphorylation (5473/T308) in LNCaP (FIG. 5A) and C4-2 (FIG. 5B) cells treated with the tested compound (BZ36), compared to non-treated cells. Significant decreases were also observed in the phosphorylation of ERK1/2 (T202/Y204, T185/Y187) and MEK1/2 (S218/5222, S222/S226) in BZ36-treated C4-2 cells as compared to control cells. Interestingly, phosphorylation of AMPKcc was increased following treatment with the tested compound (BZ36) in both LNCaP and C4-2 cancer cells (FIG. 5A-B). Western blot analysis further proved phosphorylation changes in these proteins (FIG. 5C-D). Inhibition of Akt phosphorylation was not the result of decreased expression of Akt because total Aid protein levels were similar in treated and not treated cells (FIG. 5C). In contrast to AKT, treatment with the tested compound (BZ36) induced a significant increase in phosphorylated AMPKcc in both LNCaP and C4-2 cells (FIG. 5C). Interestingly, the tested compound (BZ36) activated AMPK more efficiently than the classical AMPK activator AICAR (FIG. 5C). We next investigated the active phosphorylation status of downstream signaling proteins, such as ERK1/2. Immublot analysis revealed decreased phosphorylation of ERK1/2 in both LNCaP and C4-2 cells following exposure to the tested compound (BZ36), whereas total ERK expression was not changed (FIG. 5D). Moreover, we found that phosphorylation of GSK3a/13, which is inhibited by phosphorylation by AKT, was also abrogated by BZ36 treatment in LNCaP and C4-2 cells (FIG. 5D). These results were consistent with the abrogation of, at least AKT signaling in cells treated with the tested compound (BZ36).

AKT is activated by PIP3, which is the result of PIP2 phosphorylation by PI3K. Since SCD-1 inhibition induced a dramatic decrease in de novo synthesis of phospholipids, which are PI(3,4,5)P₃ precursors, we anticipated that treatment with the tested compound (BZ36) would have an impact in PIP3 concentration in these prostate cancer cells. ELISA test demonstrated that PI(3,4,5)P₃ concentration was strongly decreased by 84% and 92% respectively in LNCaP and C4-2 BZ36-treated, compared to non-treated cells. These results suggested that inhibition of AKT activity in these cells was mediated, at least partially by decreased synthesis of PI(3,4,5)P₃ precursors after treatment with the tested compound (BZ36).

It was also particularly interesting the effects on GSK3α/β, which is further downstream AKT pathway. Activation of GSK3α/β by SCD-1 inhibitors, resulted in the disruption of β-catenin signaling, demonstrated by the decreased activity of a β-cat reporter in response to BZ36 in both LNCaP, and C4-2 cells (FIG. 5F). This was fully consistent with changes in the expression of genes in the β-cat pathway, including, but not limited to decreased expression of several members of the frizzled family, or decreased cyclin D1, D2, D3, myc, or c-jun expression, or decreased expression of several Wnt family members in C4-2 cells treated with the tested compound (BZ36) (FIG. 5G). Taken together these results proved that SCD-1 inhibition, directly or indirectly results in a strong disruption of major signaling pathways implicated in cell proliferation, migration, and survival.

Inhibition of SCD-1 Activity by 1N-[(2-bromo-5-methoxybenzoyl)]-4N-[(phenylpropan)pyridine-3-carboxamide)]piperazine Inhibit LNCaP and C4-2 Tumor Growth In Vivo

The tested compound is the 1N-[(2-bromo-5-methoxybenzoyl)]-4N-[(phenylpropan)pyridine-3-carboxamide)] piperazine (BZ36).

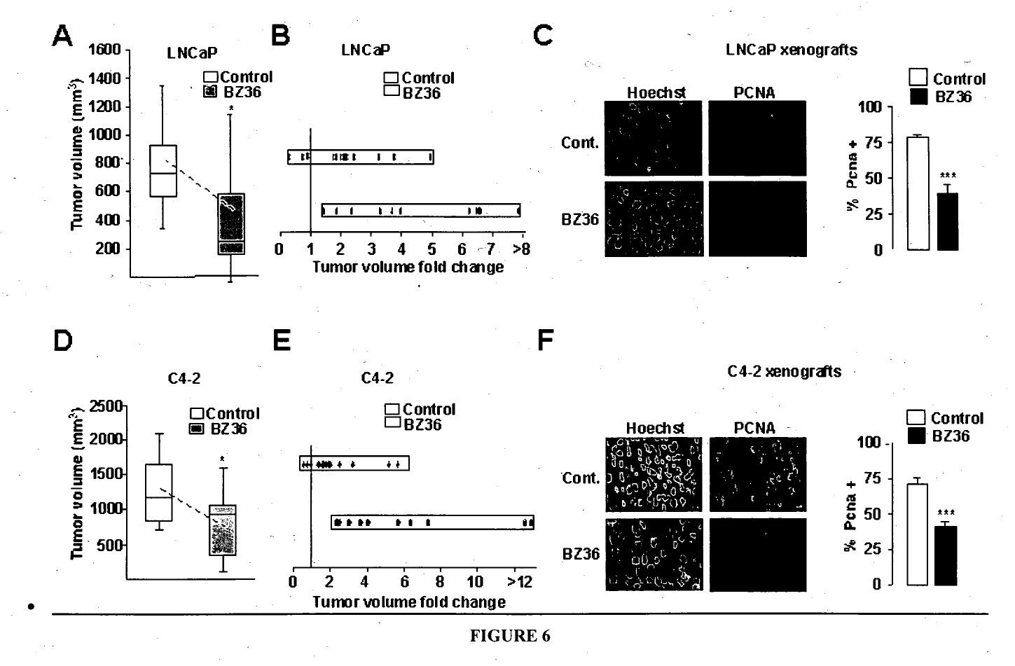

It has also been evaluated whether reduction of SCD-1 activity could inhibit the growth of CaP cells in mice. LNCaP and C4-2 cells were injected subcutaneously in male athymic nude mice. Treatment of each individual mouse started when tumor growth was measurable. In two independent experiments, it was observed that treatment of mice with 80 mg/kg of the tested compound significantly reduced both androgen-dependent LNCaP (FIGS. 6 A-B) and androgen-insensitive C4-2 (FIGS. 6 D-E) tumor volume and tumor growth rate, as compared with control mice that received vehicle only. Moreover, it was observed that these tested compound treatment at 80 mg/kg induced LNCaP and C4-2 tumor regression in 27% of LNCaP (FIG. 6B) and 19% of C4-2 (FIG. 6E) xenografted mice, whereas no tumor regression was observed in control mice. On the opposite, it was observed that LNCaP or C4-2 tumor volume was increased more than 4-fold in 42% of vehicle-treated mice, whereas this was the case in only 18% (LNCaP) and 12% (C4-2) of the tested compound-treated mice (FIGS. 6B, and E). PCNA immunostaining on tumors from LNCAP (FIG. 6C) and C4-2 (FIG. 6F) xenografts demonstrated significant decreases of respectively 50% and 41% of proliferative tumor cells in animals treated with BZ36 at 80 mg/kg compared to control animals.

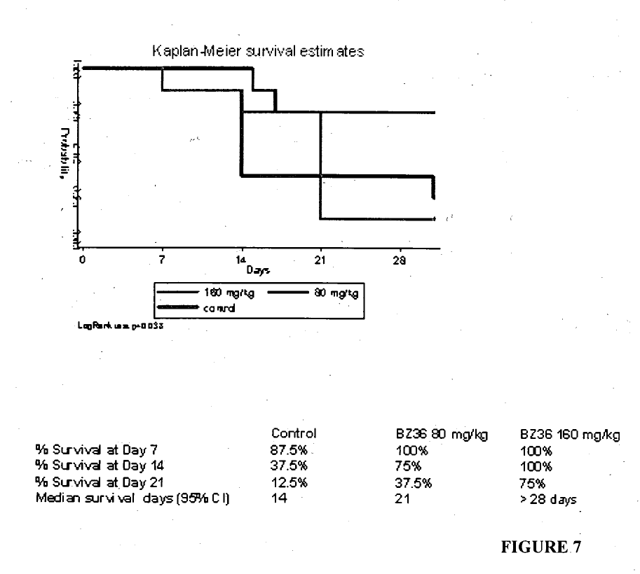

We next examined whether treatment with the tested compound (BZ36) improved the survival of mice with pre-established androgen-independent PC tumors derived from C4-2 cells. Nude mice were treated with vehicle or two doses of BZ36, 80 mg/kg or 160 mg/kg, and followed until death or until tumor volume did reach 2000 mm³. Among mice bearing subcutaneous C4-2 tumors, treatment of animals with the tested compound (BZ36) result in significant and dose-dependant prolongation of animal survival in comparison to vehicle control (p=0.038, FIG. 7). Indeed, after the treatment was started, the median survival in the control group was of 14 days although it was of 21 days in animals treated with 80 mg/kg of the tested compound (BZ36). At 14 days of treatment, 37.5% of animals did survived in the control group against 75% and 100% in the groups treated with 80 mg/kg or 160 mg/kg of the tested compound (BZ36) respectively. At 28 days of treatment, 75% of animals receiving 160 mg/kg of the tested compound (BZ36) did survived against only 12.5% in the control group.

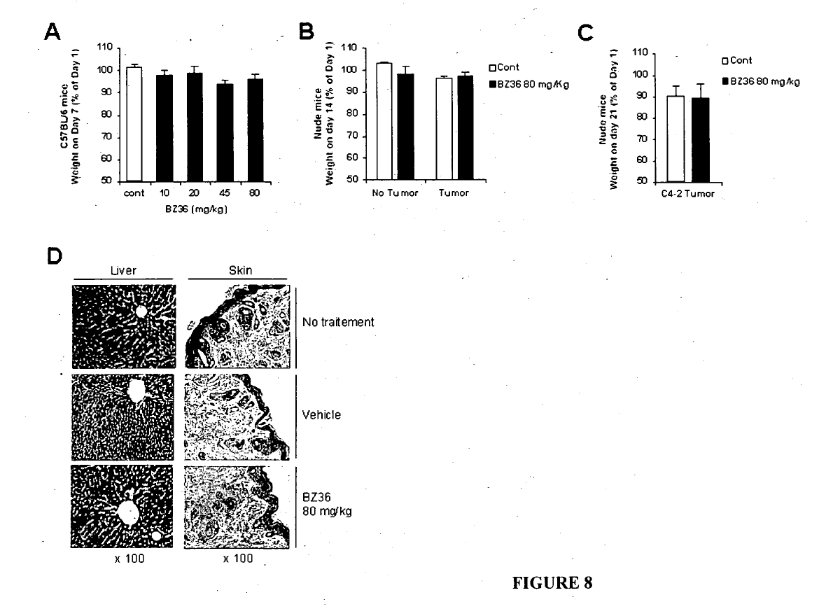

Importantly, no significant weight loss or other toxicity was observed in nude mice following daily i.p. injection of the tested compound (BZ36) (FIG. 8A-C). Histological examination confirmed the absence of toxicity in liver and skin, and no difference of tissue integrity between animals treated with vehicle or the tested compound (BZ36) (FIG. 8D).

Inhibition of SCD-Like Activities in Transformed MEFs Decreases Proliferation and Tumorigenesis in Mice

The tested compound is the 1N-[(2-bromo-5-methoxybenzoyl)]-4N-[(phenylpropan)pyridine-3-carboxamide)]piperazine (BZ36).

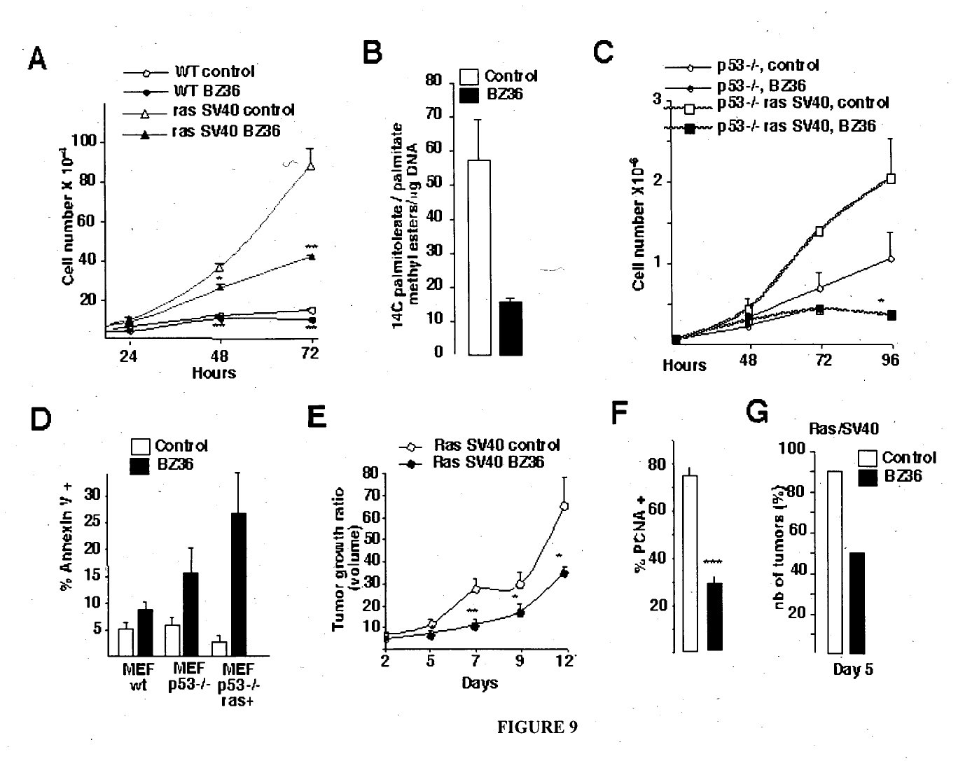

To further document the strong antiproliferative effects of SCD inhibitors on Ras-transformed cells we next tested their impact on Mouse embryonic fibroblasts (MEFs) transformed by genetically defined elements or genetic background that cooperate with Ras in transformation. Proliferation index of MEFs fully transformed by activated Ras (HaRasVl2) and SV40 Large T was strongly decreased by treatment with the tested compound (BZ36) whereas that of the slowly growing normal MEFs (WT) was only moderately reduced (FIG. 9A). Similar to prostate cancer cells, inhibition of SCDs activity in these transformed MEFs resulted in decreased lipogenesis as measured by the decrease of [¹⁴C]-palmitoleic to palmitic methyl ester acids ratio (FIG. 9B).

Importantly, the potent antiproliferative effect of BZ36 on cells containing Large T confirmed that SCD inhibition has impact on cells with altered pRB- and p53-pathways, a hallmark of most cancer cells. Consistently, the tested compound (BZ36) also efficiently blocked the proliferation of p53−/− MEFs fully transformed by HaRasVl2 (FIG. 9C). Interestingly, these antiproliferative effects of the tested compound (BZ36) were associated with a strong induction of cell death (annexin V-positive cells) in all transformed cells, including p53−/− MEFS transformed by activated Ras, suggesting that the end result of SCD inhibition in cancer cells is a p53-independent cell death (FIG. 9D).

Investigations have been also conducted to appreciate whether reduction of SCD-1 activity by the tested compound molecule would inhibit the development and the growth of HaRasV12-SV40 Large T-transformed Mefs in vivo.

When compared with vehicle injection, the tested compound inhibitor significantly prevented ras SV40 MEFs tumors growth in mice as early as day 2 after treatment. Moreover, inhibition of tumor growth by the tested compound was maintained all along the 12 days of this experiment (FIG. 9E) This experiment proved that this treatment was efficient in the presence of activated Ras, which is a common situation found in human cancers. PCNA immunostaining on tumors from ras SV40 MEFs xenografts demonstrated a significant 61% decrease of proliferative tumor cells in mice treated with the tested compound, compared to vehicle-treated mice (FIG. 9F).

Furthermore, it has also been shown that pre-treatment of mice with the tested compound one week prior tumor generation by ras SV40 MEFs, reduced tumor formation. Five days after cells were grafted, almost all of vehicle treated mice developed tumors, whereas tumors grown only in 50% of the tested compound treated mice (FIG. 9G). This suggested that Ras-mediated transformation requires active de novo lipid synthesis in order to generate cancers. Collectively, these data confirmed our observation with LNCaP and C4-2 human cancer cell lines, and demonstrate that inhibition of SCD activity has a potent inhibitory effect on the cell viability and tumorigenicity of p53-deficient and Ras-transformed cells.

Accordingly, the compounds according to the instant invention and more particularly compound of formula (I) are particularly useful for the treatment of prostate cancer.

According to the present invention, such compound of formula I could be particularly useful in combination with standard therapy, in any clinical situation in which a strong response is expected from standard therapy, and in which, nevertheless, relapses are frequent.

By “another anti-cancer treatment” is meant any other suitable treatment approved for cancer treatment. In particular, said other anti-cancer treatment may be selected from the group consisting of chemotherapy, surgery, radiotherapy, hormonotherapy, and/or immunotherapy.

In the case of chemotherapy, at least one secondary chemotherapeutic agent may be used. Such an agent may be selected from the group consisting of growth inhibitory agents (defined as compounds or compositions which inhibit growth of a cell, either in vitro or in vivo) and cytotoxic agents (defined as compounds or compositions which inhibits or prevents the function of cells and/or causes destruction of cells).

In a particular embodiment of the invention, a secondary chemotherapeutic agent may be selected from the group consisting of taxanes, topoisomerase II inhibitors, DNA alkylating agents, anti-metabolite, anti-tubuline, vinca alkaloids, intercalating agents and platinium salts.

In a more particular embodiment of the invention, a secondary chemotherapeutic agent may be selected from the group consisting of: paclitaxel, docetaxel, doxorubicin, epirubicin, daunorubicin, etoposide, bleomycin, tamoxifen, prednisone, dacarbazine, mechlorethamine, methotrexate, 5-fluorouracil, anthracyclines, adriamicin, vinblastine, vincristine, vinorelbine, topotecan, carboplatin, cisplatin, permetrexed, irinotecan, gemcitabine, gefinitib, erlotinib, fludarabin, ifosfamide, procarbazine, mitoxanthrone, melphalan, mitomycin C, chlorambucil, cyclophosphamide and platinium salts.

Of course, any suitable combination of chemotherapeutic drugs may be used, depending on the type of cancer.

Compounds of formule IIa), pharmaceutical compositions and therapeutic methods based on such compounds are particularly useful for the treatment and/or prevention of diseases mediated by stearoyl-CoA desaturase (SCD), especially human SCD (hSCD), by administering to a patient in need of such treatment an effective amount of an SCD-inhibiting, agent.

An SCD-mediated disease or condition also includes metabolic syndrome (including but not limited to dyslipidemia, obesity and insulin resistance, hypertension, microalbuminemia, hyperuricaemia, and hypercoagulability), Syndrome X, diabetes, insulin resistance, decreased glucose tolerance, non-insulin-dependent diabetes mellitus, Type II diabetes, Type I diabetes, diabetic complications, body weight disorders, weight loss, body mass index and leptin related diseases.

As used herein, the term “metabolic syndrome” is a recognized clinical term used to describe a condition comprising combinations of Type II diabetes, impaired glucose tolerance, insulin resistance, hypertension, obesity, increased abdominal girth, hypertriglyceridemia, low HDL, hyperuricaemia, hypercoagulability and/or microalbuminemia.

An SCD-mediated disease or condition also includes fatty liver, hepatic steatosis, hepatitis, non-alcoholic hepatitis, non-alcoholic steatohepatitis (NASH), alcoholic hepatitis, acute fatty liver, fatty liver of pregnancy, drug-induced hepatitis, erythrohepatic protoporphyria, iron overload disorders, hereditary hemochromatosis, hepatic fibrosis, hepatic cirrhosis, hepatoma and conditions related thereto.

An SCD-mediated disease or condition also includes but is not limited to a disease or condition which is, or is related to primary hypertriglyceridemia, or hypertriglyceridemia secondary to another disorder or disease, such as hyperlipoproteinemias, familial histiocytic reticulosis, lipoprotein lipase deficiency, apolipoprotein deficiency (such as ApoCII deficiency or ApoE deficiency), and the like, or hypertriglyceridemia of unknown or unspecified ethio logy.

An SCD-mediated disease or condition also includes a disorder of polyunsaturated fatty acid (PUFA) disorder, or a skin disorder, including but not limited to eczema, acne, psoriasis, keloid scar formation or prevention, diseases related to production or secretions from mucous membranes, such as monounsaturated fatty acids, wax esters, and the like. An SCD-mediated disease or condition also includes inflammation, sinusitis, asthma, pancreatitis, osteoarthritis, rheumatoid arthritis, cystic fibrosis, and pre-menstrual syndrome.

An SCD-mediated disease or condition also includes but is not limited to a disease or condition which is, or is related to cancer, more particularly lung cancer and prostate cancer, breast cancer, hepatomas and the like, neoplasia, malignancy, metastases, tumours (benign or malignant), carcinogenesis.

An SCD-mediated disease or condition also includes a condition where increasing lean body mass or lean muscle mass is desired, such as is desirable in enhancing performance through muscle building. Myopathies and lipid myopathies such as carnitine palmitoyltransferase deficiency (CPT I or CPT II) are also included herein. Such treatments are useful in humans and in animal husbandry, including for administration to bovine, porcine or avian domestic animals or any other animal to reduce triglyceride production and/or provide leaner meat products and/or healthier animals.

An SCD-mediated disease or condition also includes a disease or condition which is, or is related to, neurological diseases, psychiatric disorders, multiple sclerosis, eye diseases, and immune disorders.

1. An inhibitor of an activity of stearoyl-CoA-desaturase-1 (SCD-1) enzyme for treatment of prostate cancer, wherein said inhibitor of activity of SCD-1 is a compound of formula (I): 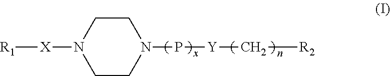
wherein:
X represents —CO— or —SO₂—;
R₁ represents an alkyl, a cycloalkyl, an aryl or a heteroaryl group in C₅ to C₁₄, said aryl or heteroaryl being optionally substituted with one or more groups Rₐ;
Rₐ represents a halogen atom, a hydroxyl group, —NO₂, —CN, —NH₂, —N(C₁₋₆alkyl)₂, a C₁₋₆alkyl, a C₁₋₆alkoxy, a —C(O)—C₁₋₆alkyl, C₂₋₆alkenyl, C₃₋₆cycloalkyl, aryl, C₃₋₆ heterocyclyl or heteroaryl, said alkyl, alkoxy, alkenyl, cycloalkyl, aryl, heterocyclyl or heteroaryl being optionally substituted with one or more halogen atom, C₁₋₆ alkyl, C₁₋₆ alkoxy, —C(O)—C₁₋₆ alkyl, —NO₂, —CF₃, —OCF₃, —CN, —NH₂, and/or —N(C₁₋₆alkyl)₂;
P is a heteroaromatic cycle in C₅ to C₁₄;
x represents 0 or 1;
Y represents —SO₂— or *—CO—NH— or *—CS—NH—, with * indicating a link to —(P)ₓ—;
n represents 0, 1, 2 or 3;
R₂ represents an alkyl, a cycloalkyl, an aryl or a heteroaryl group in C₅ to C₁₄, said aryl or heteroaryl being optionally substituted with one or more groups R_{b};
R_{b} represents a halogen atom, a hydroxyl group, NO₂, —CN, —CF₃, —OCF₃, a C₁₋₆ alkyl, C₁₋₆ alkoxy, —C(O)—C₁₋₆ alkyl, —NH₂, —N(C₁₋₆alkyl)₂, C₃₋₆ cycloalkyl, C₃₋₆ heterocyclyl, aryl, heteroaryl, said alkyl, alkoxy, cycloalkyl, heterocyclyl, aryl, heteroaryl being optionally substituted with one or more hydroxyl group, —CF₃, —OCF₃, —NH₂, —NO₂, and/or —CN,
or one of its salts or enantiomer forms.

2. The inhibitor according to claim 1, wherein the prostate cancer is a prostate cancer with a Gleason score equal or superior to 7.

3. The inhibitor according to claim 1, wherein X represents —CO—.

4. The inhibitor according to claim 1, wherein R1 is an aryl or a heteroaryl group in C₅ to C₁₄, said aryl or heteroaryl being optionally substituted with one or more groups Rₐ.

5. The inhibitor according to claim 1, wherein Y represents —SO₂— or * —CO—NH—.

6. The inhibitor according to claim 1, wherein said compound is 1N-[(2-bromo-5-methoxybenzoyl)]-4N-[(phenylpropan)pyridine-3-carboxamide)] piperazine.

7. The inhibitor according to claim 1, wherein said inhibitor is of formula (IIa): 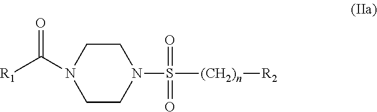
wherein R₁, R₂ and n are as defined in claim 1.

8. The inhibitor according to claim 1, wherein R₁ represents a phenyl group, optionally substituted with one or more Rₐ, said Rₐ representing a halogen atom or a C₁₋₆ alkoxy group.

9. The inhibitor according to claim 1, wherein R₁—(C═O) represents a heteroarylcarbonyl group selected from the group consisting of nicotinoyl, thiophene carbonyle, and thiazolecarbonyle.

10. The inhibitor according to claim 1, wherein R₂ is a difluorophenyl group.

11. The inhibitor according to claim 1, wherein said inhibitor is selected from the group consisting of:
1N-[(2-bromo-5-methoxybenzoyl)]-4N-[(phenyl)sulfonyl]piperazine,
1N-[(2-bromo-5-methoxybenzoyl)]-4N-[(4-methylphenyl)sulfonyl]piperazine,
1N-[(2-bromo-5-methoxybenzoyl)]-4N-[(4-propylphenyl)sulfonyl]piperazine,
1N-[(2-bromo-5-methoxybenzoyl)]-4N-[(4-isopropylphenyl)sulfonyl]piperazine,
1N-[(2-bromo-5-methoxybenzoyl)]-4N-[(4-nitrophenyl)sulfonyl]piperazine,
1N-[(2-bromo-5-methoxybenzoyl)]-4N-[(4-chlorophenyl)sulfonyl]piperazine,
1N-[(2-bromo-5-methoxybenzoyl)]-4N-[(4-bromophenyl)sulfonyl]piperazine,
1N-[(2-bromo-5-methoxybenzoyl)]-4-[(4-fluorophenyl)sulfonyl]piperazine,
1N-[(2-bromo-5-methoxybenzoyl)]-4N-[(4-methoxyphenyl]sulfonyl]piperazine,
1N-[(2-bromo-5-methoxybenzoyl)]-4N-[(4-acetylphenyl)sulfonyl]piperazine,
1N-[(2-bromo-5-methoxybenzoyl)]-4N-[(phenylmethane)sulfonyl]piperazine,
1N-[(2-bromo-5-methoxybenzoyl)]-4N-[(4-biphenyl)sulfonyl]piperazine,
1N-[(2-bromo-5-methoxybenzoyl)]-4N-[(2-fluorophenyl)sulfonyl]piperazine,
1N-[(2-bromo-5-methoxybenzoyl)]-4N-t(3-fluorophenyl)sulfonyl]piperazine,
1N-[(2-bromo-5-methoxybenzoyl)]-4N-[(4-trifluoromethylphenyl)sulfonyl]piperazine,
1N-[(2-bromo-5-methoxybenzoyl)]-4N-[(4-trifluoromethoxyphenyl)sulfonyl]piperazine,
1N-[(2-bromo-5-methoxybenzoyl)]-4N-[(3-trifluoromethylphenyl)sulfonyl]piperazine,
1N-[(2-bromo-5-methoxybenzoyl)]-4N-[(2-trifluoromethylphenyl)sulfonyl]piperazine,
1N-[(2-bromo-5-methoxybenzoyl)]-4N-[(2,4-difluorophenyl)sulfonyl]piperazine,
1N-[(2-bromo-5-methoxybenzoyl)]-4N-[(2,6-difluorophenyl)sulfonyl]piperazine,
1N-[(2-bromo-5-methoxybenzoyl)]-4N-[(2-thiophene)sulfonyl]piperazine,
1N-[(2-bromo-5-methoxybenzoyl)]-4N-[(3-thiophene)sulfonyl]piperazine,
1N-[(2-bromo-5-methoxybenzoyl)]-4N-[(3-furan)sulfonyl]piperazine,
1N-[(2-bromo-5-methoxybenzoyl)]-4N-[(2-furan)sulfonyl]piperazine,
1N-[(2-bromo-5-methoxybenzoyl)]-4N-[1-methyl-1H-imidazole)sulfonyl]piperazine,
1N-[(3-bromo-4-methoxybenzoyl)]-4-N-[(2,4-difluorophenyl)sulfonyl]piperazine,
1N-[(2-methoxy-5-bromobenzoyl)]-4N-[(2,4-difluorophenyl)sulfonyl]piperazine,
1N-[(2-methoxy-3-bromobenzoyl)]-4-N-[(2,4-difluorophenyl)sulfonyl]piperazine,
1N-[(4-methoxybenzoyl)]-4-N-[(2,4-difluorophenyl)sulfonyl]piperazine,
1N-[(2-methoxybenzoyl)]-4-N-[(2,4-difluorophenyl)sulfonyl]piperazine,
1N-[(3-methoxybenzoyl)]-4-N-[(2,4-difluorophenyl)sulfonyl]piperazine,
1N-[nicotinoyl]-4-N-[(2,4-difluorophenyl)sulfonyl]piperazine,
1N-[isonicotinoyl]-4-N-[(2,4-difluorophenyl)sulfonyl]piperazine,
1N-[picolinoyl]-4-N-[(2,4-difluorophenyl)sulfonyl]piperazine,
1N-[3-thiophenecarbonyl]-4-N-[(2,4-difluorophenyl)sulfonyl]piperazine,
1N-[4-thiazolecarbonyl]-4-N-[(2,4-difluorophenyl)sulfonyl]piperazine,
1N-[(4-bromobenzoyl)]-4-N-[(2,4-difluorophenyl)sulfonyl]piperazine, and their salts.

12. A compound of formula (IIa): 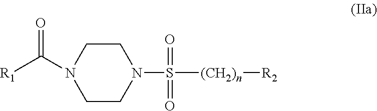
wherein:
R₁ represents an alkyl, an aryl or a heteroaryl group in C₅ to C₁₄, said aryl or heteroaryl being optionally substituted with one or more groups Rₐ;
Rₐ represents a halogen atom, a hydroxyl group, —NO₂, —CN, —NH₂, —N(C₁₋₆alkyl)₂, a C₁₋₆alkyl, a C₁₋₆alkoxy, a —C(O)—C₁₋₆alkyl, C₂₋₆alkenyl, C₃₋₆cycloalkyl, aryl, C₃₋₆ heterocyclyl or heteroaryl, said alkyl, alkoxy, alkenyl, cycloalkyl, aryl, heterocyclyl or heteroaryl being optionally substituted with one or more halogen atom, C₁₋₆ alkyl, C₁₋₆ alkoxy, —C(O)—C₁₋₆ alkyl, —NO₂, —CF₃, —OCF₃, —CN, —NH₂, and/or —N(C₁₋₆alkyl)₂;
R₂ is a difluorophenyl group, and
n is 0, 1, 2, or 3.

13. A compound selected from the group consisting of:
1N [(2-bromo-5-methoxybenzoyl)]-4N-[(phenyl)sulfonyl]piperazine,
1N-[(2-bromo-5-methoxybenzoyl)]-4N-[(4-methylphenyl)sulfonyl]piperazine,
1N-[(2-bromo-5-methoxybenzoyl)]-4N-[(4-propylphenyl)sulfonyl]piperazine,
1N-[(2-bromo-5-methoxybenzoyl)]-4N-[(4-isopropylphenyl)sulfonyl]piperazine,
1N-[(2-bromo-5-methoxybenzoyl)]-4N-[4-nitrophenyl)sulfonyl]piperazine,
1N-[(2-bromo-5-methoxybenzoyl)]-4N-[(4-chlorophenyl)sulfonyl]piperazine,
1N-[(2-bromo-5-methoxybenzoyl)]-4N-[(4-bromophenyl)sulfonyl]piperazine,
1N-[(2-bromo-5-methoxybenzoyl)]-4-[(4-fluorophenyl)sulfonyl]piperazine,
1N-[(2-bromo-5-methoxybenzoyl)]-4N-[(4-methoxyphenyl)sulfonyl]piperazine,
1N-[(2-bromo-5-methoxybenzoyl)]-4N-[(4-acetylphenyl)sulfonyl]piperazine,
1N-[(2-bromo-5-methoxybenzoyl)]-4N-[(phenylmethane)sulfonyl]piperazine,
1N-[(2-bromo-5-methoxybenzoyl)]-4N-[(4-biphenyl)sulfonyl]piperazine,
1N-[(2-bromo-5-methoxybenzoyl)]-4N-[(2-fluorophenyl)sulfonyl]piperazine,
1N-[(2-bromo-5-methoxybenzoyl)]-4N-[(3-fluorophenyl)sulfonyl]piperazine,
1N-[(2-bromo-5-methoxybenzoyl)]-4N-[(4-trifluoromethylphenyl)sulfonyl]piperazine,
1N-[(2-bromo-5-methoxybenzoyl)]-4N-[(4-trifluoromethoxyphenyl)sulfonyl]piperazine,
1N-[(2-bromo-5-methoxybenzoyl)]-4N-[(3-trifluoromethylphenyl)sulfonyl]piperazine,
1N-[(2-bromo-5-methoxybenzoyl)]-4N-[(2-trifluoromethylphenyl)sulfonyl]piperazine,
1N-[(2-bromo-5-methoxybenzoyl)]-4N-[(2,4-difluorophenyl)sulfonyl]piperazine,
1N-[(2-bromo-5-methoxybenzoyl)]-4N-[(2,6-difluorophenyl)sulfonyl]piperazine,
1N-[(2-bromo-5-methoxybenzoyl)]-4N-[(2-thiophene)sulfonyl]piperazine,
1N-[(2-bromo-5-methoxybenzoyl)]-4N-[(3-thiophene)sulfonyl]piperazine,
1N-[(2-bromo-5-methoxybenzoyl)]-4N-[(3-furan)sulfonyl]piperazine,
1N-[(2-bromo-5-methoxybenzoyl)]-4N-[(2-furan)sulfonyl]piperazine,
1N-[(2-bromo-5-methoxybenzoyl)]-4N-[(1-methyl-1H-imidazole)sulfonyl]piperazine,
1N-[(3-bromo-4-methoxybenzoyl)]-4-N-[(2,4-difluorophenyl)sulfonyl]piperazine,
1N-[(2-methoxy-5-bromobenzoyl)]-4N-[(2,4-di fluorophenyl) sulfonyl] piperazine,
1N-[(2-methoxy-3-bromobenzoyl)]-4-N-[(2,4-di fluorophenyl)sulfonyl]piperazine,
1N-[(4-methoxybenzoyl)]-4-N-[(2,4-difluorophenyl)sulfonyl]piperazine,
1N-[(2-methoxybenzoyl)]-4-N-[(2,4-difluorophenyl)sulfonyl]piperazine,
1N-[(3-methoxybenzoyl)]-4-N-[(2,4-difluorophenyl)sulfonyl]piperazine,
1N-[nicotinoyl]-4-N-[(2,4-difluorophenyl)sulfonyl]piperazine,
1N-[isonicotinoyl]-4-N-[(2,4-difluorophenyl)sulfonyl]piperazine,
1N-[picolinoyl]-4-N-[(2,4-difluorophenyl)sulfonyl]piperazine,
1N-[3-thiophenecarbonyl]-4-N-[(2,4-difluorophenyl) sulfonyl]piperazine,
1N-[4-thiazolecarbonyl]-4-N-[(2,4-difluorophenyl)sulfonyl]piperazine,
1N-[(4-bromobenzoyl)]-4-N-[(2,4-difluorophenyl)sulfonyl]piperazine, and their salts.

14. A pharmaceutical composition containing as an active agent at least one compound according to claim 12.
